(19) United States
(12) Patent Application Publication
(10) Pub. No.: US 2025/0387184 A1
(43) Pub. Date: Dec. 25, 2025

(54) USER INTERFACE FOR IMAGE GUIDED SURGICAL ROBOTIC PLATFORM

(71) Applicant: Andromeda Surgical, Inc., San Francisco, CA (US)

(72) Inventors: Kartik Tiwari, San Jose, CA (US); Mike Lewis, Walnut Creek, CA (US)

(73) Assignee: Andromeda Surgical, Inc., San Francisco, CA (US)

(21) Appl. No.: 19/245,002

(22) Filed: Jun. 20, 2025

Related U.S. Application Data
(60) Provisional application No. 63/662,378, filed on Jun. 20, 2024.

(51) Int. Cl. A61B 34/35; A61B 18/00; A61B 18/22; A61B 34/00; A61B 34/30; G06F 3/04886

(52) U.S. Cl. CPC A61B 34/35; A61B 34/25; A61B 2018/00547; A61B 18/22; A61B 2034/301; G06F 3/04886

(57) ABSTRACT

A system includes a surgical robot, including a robot arm configured to hold an end effector and at least one circuit configured to receive imaging data of a surgical site and generate a presentation to be presented on a user interface. The presentation includes imaging data of the surgical site, a map of an anatomy of the surgical site surrounding the imaging data, and a first designated touch area. The at least one circuit is also configured to receive a user input on the user interface and actuate the robot arm to move the end effector in one of a depth direction or a planar direction based on the user input.

### CROSS REFERENCE TO RELATED APPLICATION(S)

This application claims priority to and the benefit of U.S. Provisional Application No. 63/662,378, filed Jun. 20, 2024, the contents of which are incorporated herein by reference in its entirety.

### FIELD

This disclosure relates to processes and systems for an image-guide surgical robotic platform.

### BACKGROUND

Various surgical techniques are known to provide high degree of successful outcomes when executed properly. Nonetheless, the high degree of manual surgical precision and expertise associated with such techniques may prevent the techniques from being widely adopted.

For instance, holmium laser enucleation of the prostate (HoLEP) is a trans-urethral procedure utilizing a Ho:YAG laser fiber to enucleate the adenoma of the prostate. The enucleated adenoma is then removed via morcellation from the bladder cavity. HoLEP was developed by Dr. Peter Gilling in the 1990s and has become a first-line surgical treatment for benign prostatic hypertrophy (BPH). Decades of data demonstrate HoLEP's safety and superiority in surgical outcomes compared to alternative treatments. However, the procedure's difficult learning curve remains an obstacle to its widespread adoption. There remains a need for methods and systems to make the HoLEP procedure more accessible to surgeons and lower the learning and training curve associated with the procedure.

### SUMMARY

The present disclosure relates to a system, including: a surgical robot, including a robot arm configured to hold an end effector; and at least one circuit configured to: generate a presentation to be presented on a user interface, wherein the presentation includes: imaging data of a surgical site; a map of an anatomy of the surgical site surrounding the imaging data; and a first designated touch area; receive a user input on the user interface; and actuate the robot arm to move the end effector in one of a depth direction or a planar direction based on the user input. In some embodiments, the at least one circuit is configured to receive imaging data of the surgical site.

The present disclosure relates to a system, including: a surgical robot, including a robot arm configured to hold an end effector; and at least one circuit configured to: generate a presentation to be presented on a user interface, wherein the presentation includes: imaging data of a surgical site; and a map of an anatomy of the surgical site surrounding the imaging data; receive a user input on the user interface; and actuate the robot arm to move the end effector with a speed based on the user input. In some embodiments, the at least one circuit is configured to receive imaging data of the surgical site.

The present disclosure relates to a method, including: receiving imaging data of a surgical site, wherein a robot arm is configured to position an end effector in the surgical site; generating a presentation to be presented on a user interface, wherein the presentation includes: imaging data of the surgical site; a map of an anatomy of the surgical site surrounding the imaging data; and a first designated touch area; receiving a user input on a user interface; and actuating the robot arm to move the end effector in one of a depth direction or a planar direction based on the user input.

The present disclosure relates to a method, including: receiving imaging data of a surgical site, wherein a robot arm is configured to position an end effector in the surgical site; generating a presentation to be presented on a user interface, wherein the presentation includes: imaging data of the surgical site; and a map of an anatomy of the surgical site surrounding the imaging data; receiving a user input on the user interface; and actuating the robot arm to move the end effector with a speed based on the user input.

The present disclosure relates to at least one non-transitory computer-readable storage medium having encoded thereon executable instructions that, when executed by at least one processor, cause the at least one processor to: receive imaging data of a surgical site, wherein a robot arm is configured to position an end effector in the surgical site; generate a presentation to be presented on a user interface, wherein the presentation includes: imaging data of the surgical site; a map of an anatomy of the surgical site surrounding the imaging data; and a first designated touch area; receive a user input on a user interface; and actuate the robot arm to move the end effector in one of a depth direction or a planar direction based on the user input.

The present disclosure relates to at least one non-transitory computer-readable storage medium having encoded thereon executable instructions that, when executed by at least one processor, cause the at least one processor to: receive imaging data of a surgical site, wherein a robot arm is configured to position an end effector in the surgical site; generate a presentation to be presented on a user interface, wherein the presentation includes: imaging data of the surgical site; and a map of an anatomy of the surgical site surrounding the imaging data; receive a user input on the user interface; and actuate the robot arm to move the end effector with a speed based on the user input.

The present disclosure relates to a system, including: a surgical robot, including a robot arm configured to hold an end effector; and one or more processing devices configured to: receive an estimate of a position or a location of the end effector in a surgical site; receive an estimate of a depth of tissue in front of the end effector; generate a map of the surgical site, wherein the map extends beyond a field of view of real-time imaging data of the surgical site generated at a current position or location of the end effector; and present the map of the surgical site and the real-time imaging data on a display.

The present disclosure relates to a system, including: a surgical robot, including a robot arm configured to hold an end effector; and one or more processing devices configured to: receive an estimate of a depth of tissue in front of the end effector; receive input to move the end effector in first and second directions; generate an instruction to move the end effector in first and second directions in accordance with the received input; and automatically generate an instruction to move the end effector in a third direction.

### BRIEF DESCRIPTION OF THE DRAWINGS

The present disclosure is further described in the detailed description which follows, in reference to the noted plurality of drawings by way of non-limiting examples of exemplary embodiments, in which like reference numerals represent similar parts throughout the several views of the drawings, and wherein:

FIG. 1 depicts an illustrative system for guiding a surgeon during a medical procedure, according to one or more embodiments herein;

FIG. 2 depicts an illustrative robotic arm of a robotic cart, according to one or more embodiments herein;

FIG. 3 depicts illustrative internal components of the robotic cart, according to one or more embodiments herein;

FIG. 4 depicts an illustrative console cart, according to one or more embodiments herein;

FIG. 5 depicts illustrative internal components of the console cart, according to one or more embodiments herein;

FIG. 6 depicts an illustrative method of presenting a map of a surgical site, according to one or more embodiments herein;

FIG. 7 depicts an illustrative example of a user interface, according to one or more embodiments herein;

FIG. 8 depicts an illustrative method of moving an end effector based on input received from a user, according to one or more embodiments herein;

FIG. 9 depicts an illustrative example of a user interface, according to one or more embodiments herein;

FIG. 10 depicts an illustrative example of a user interface, according to one or more embodiments herein;

FIG. 11 depicts an illustrative method of moving an end effector based on input received from a user, according to one or more embodiments herein;

FIG. 12 depicts an illustrative method of moving an end effector based on input received from a user, according to one or more embodiments herein;

FIG. 13 depicts an illustrative method of moving an end effector based on input received from a user, according to one or more embodiments herein;

FIG. 14 depicts an illustrative method of controlling the movement of an end effector based on input received from a user, according to one or more embodiments herein;

FIG. 15 depicts an illustrative example of controlling the movement of an end effector based on input received from the user, according to one or more embodiments herein; and

FIG. 16 depicts is a block diagram of an example computing system for controlling a robotic arm that can be used in performing surgical techniques.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

The efficacy of surgical procedures has traditionally been largely dependent on a particular surgeon's skill level and experience with a specific surgery. That is, a surgeon may need to conduct a certain number of surgeries of a particular kind (for instance holmium laser enucleation of the prostate (HoLEP)) before the surgeon becomes proficient in the surgery such that desirable and precise surgical outcomes can be expected with a high degree of certainty. Even then, however, the outcome of surgical procedures has been limited by human error of the surgeon. Such errors can be in decision making and physical precision (e.g., physical manipulation of a surgical instrument). Human surgical error, in many cases, has been tied to shortcomings in the surgeon's ability to accurately visualize the surgical target area in the patient's body. While real-time medical imaging has been beneficial, particularly, in non-invasive surgeries, such images can still be misinterpreted by a surgeon during the medical procedure. For instance, surgeons can misinterpret or get “lost” in the real-time imaging data because a narrow field of view of the imaging data is presented to the surgeon. Moreover, current robotic surgical platforms can be cumbersome for a surgeon to interact with and control. For instance, the surgical robot, and particularly the end effector controlled by the surgical robot must be moved in three dimensions. However, the imaging data is often presented in two dimensions to the surgeon, making it difficult for the surgeon to perceive the three-dimensional space of the surgical site and provide instructions for moving the end effector in three dimensions.

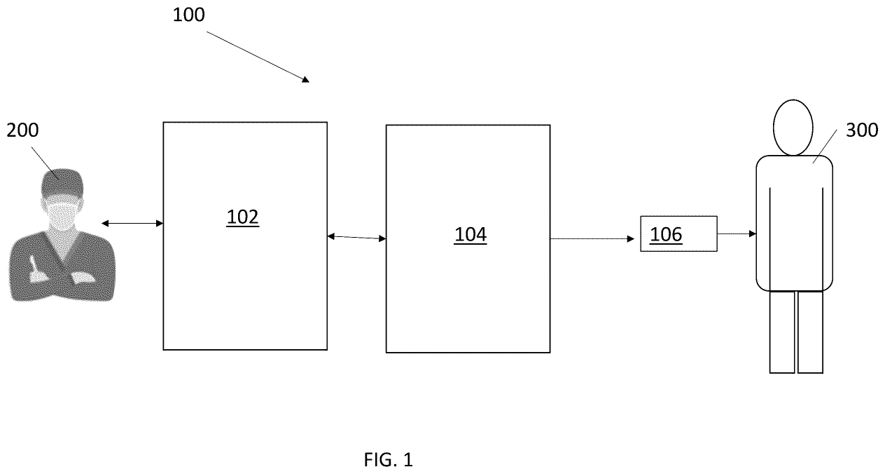

The present disclosure provides systems and methods for increasing the efficacy and reproducibility of surgical procedures by way of an image-guided surgical robotic platform. The presently disclosed surgical robotic platforms can be semi-autonomous, with varying degrees of human control or supervision over the surgical procedure, as discussed below. Referring to FIG. 1, a system 100 of the present disclosure is generally depicted. In some embodiments, the system 100 can generally include a console cart 102 and a robotic cart 104. In some embodiments, the console cart 102 and the robotic cart 104 can be in wired or wireless communication with each other such that data, information, commands, and instructions can be bi-directionally transmitted between the console cart 102 and the robot cart 104.

The robotic cart 104, as discussed in greater detail below, can generally include a movable robotic arm and an adapter positioned on the robotic arm and configured to hold and manipulate an end effector 106 (e.g. a surgical tool). In some embodiments, the adapter is fitted onto the robotic arm to hold endourology instruments and accessories needed to perform HoLEP. In some embodiments, the robotic cart 104 can include electronic systems to power and operate the robotic arm and end effector 106 and to process real time data. The end effectors 106 can be positioned relative a patient 300 and controlled to perform a desired surgical procedure, such as HoLEP. While HoLEP is discussed herein, it should be appreciated that this is merely an example, and the systems and methods discussed herein can relate to any number of surgical procedures.

The console cart 102, as discussed in greater detail below, can generally include one or more displays or user interfaces. such as input/output devices, for interacting with a surgeon 200. In some embodiments, the console cart 102 can receive data from the robotic cart 104 relating to the location (e.g., at least one of the position or the orientation) or operation of the end effector 106. In some embodiments, the console cart 102 can process the data received from the robotic cart 104. In some embodiments, the console cart 102 can process the data received from the robotic cart 104 in relation to a database of information generated from previously executed surgeries of the same type (e.g., previously executed HoLEP surgeries if the robotic cart 104 is being used to perform HoLEP). In some embodiments, the console cart 102 can present the processed data to the surgeon 200 on the one or more displays or user interfaces. In some embodiments, the console cart 102 can determine a proposed next surgical action for the robotic cart 104 to take and present, as a suggestion, the proposed next surgical action to the surgeon via the one or more displays. In some embodiments, the console cart 102 can receive an instruction from the surgeon via the one or more user interfaces, the instruction including a command for a next surgical action (e.g., movement of the end effector 106, actuation of the end effector 106, etc.) for the robotic cart 104 to take. The console cart 102 can transmit the command to the robotic cart 104.

The system 100, in presenting processed data to the surgeon 200 or in presenting a proposed next surgical action, can assist the surgeon 200 through the surgical procedure such that errors related to surgeon decision making are reduced or eliminated. The system 100, in controlling end effector 106 manipulation and activation via a robotic assembly, can more precisely execute the surgical procedure such that errors related to the surgeon 200 physically performing one or more surgical steps are reduced or eliminated. That is, in traditional surgical environment, the surgeon 200 directly interacts with the end effector 106, which is used to perform an action on the patient 300. The system 100, including the console cart 102 and the robotic cart 104, is functionally inserted between the surgeon 200 and the end effector 106 to assist the surgeon in controlling the end effector 106 to complete a surgical action on the patient 300.

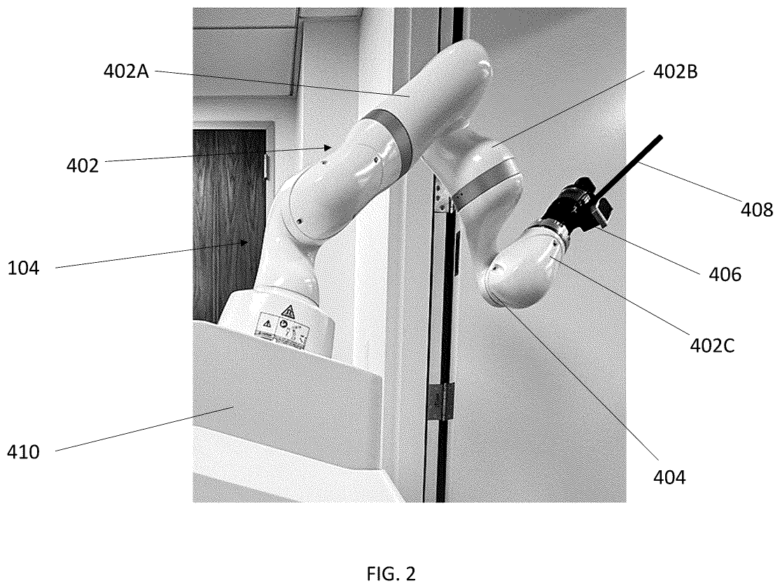

Referring now to FIG. 2, the robotic cart 104 is depicted according to some embodiments. In some embodiments, the robotic cart 104 includes a base 410 and one or more robotic arms 402 which extend from the base 410. In some embodiments, the base 410 is movable, such that the base 410) and the robotic cart 104 can be optimally positioned for performing a surgery (e.g., next to a patient, operating table, hospital bed, etc.). For instance, in some embodiments, the base 410 can include one or more wheels to enable selective positioning of the base 410. In some embodiments, the base 410 is integral with one or more other pieces of hospital equipment, such as an operating table.

In some embodiments, each of the one or more robotic arms 402 can include a plurality of arm segments 402A, 402B, 402C. Each arm segment 402A-C can be coupled to an adjacent arm segment 402A-C by a joint 404. Each joint 404 can be selectively designed to impart a desired degree of freedom between the arm segments linked by the joint 404. For instance, the joint 404 can impart the robotic arm with two, four, or six degrees of freedom between the arm segment 402c and the arm segment 402B. Generally, the one or more robotic arms 402 can be designed and controlled to have any desirable axial, angular, or rotational motion. In some embodiments, the robotic arm 402 can have seven degrees of freedom.

In some embodiments, the distalmost (e.g., nearest to the surgical site) arm segment of the robotic arm 402 (e.g., the arm segment 402C in the embodiment of FIG. 2), can include an adapter 406 at its distal end. In some embodiments, the adapter 406 can be a hardware interface configured to receive or hold one or more end effectors 408. In some embodiments, the adapter 406 can allow the one or more robotic arms 402 to grip the one or more end effectors 408, can allow for non-permanent coupling of the one or more robotic arms 402 or other portion of the robotic cart 104 with the one or more end effectors 408, and the like.

In some embodiments, the end effector 408 can be any tool or device used to perform the surgical procedure. In some embodiments, the end effector 408 can be any tool or device positioned at or near the surgical site for and during the surgical procedure. In some embodiments, the adapter 406 can be configured to hold or receive multiple end effectors 408 at once. In some embodiments, the end effector 408 can be gripped or held by the adapter 406 at the distal ends of the one or more robotic arms 402. In some embodiments, the end effector 408 can be non-fixedly coupled to the adapter 406 at the distal ends of the one or more robotic arms 402. In some embodiments, a first end effector 408 can be removed from the adapter 406 and a second. different end effector 408 can be received by the adapter 406. as required.

In some embodiments, the end effector 408 is a procedure-specific tool, such as a tool designed for or implemented in a specific surgical procedure, such as HoLEP. In some embodiments, the end effector is a procedure-generic tool, such as a tool designed for or implemented in multiple different surgical procedures. In some embodiments, the end effector 408 can plug directly into the robotic cart 104 for providing energy to the end effector 408. In some embodiments, the end effector 408 can include one or more of a cystoscope, a rigid cystoscope, an endoscope, a resectoscope, a nephroscope, a cystoscopy, resectoscopy or nephroscopy sheath, a holmium, thulium, greenlight, or blue light laser, a laser fiber, a morcellator, an irrigation system, or an aspirator. In some embodiments, the end effector 408 can be any endourology instruments and accessories needed to perform HoLEP. In some embodiments, the robotic cart 104 can supply one or more operational sources to the end effector 408, such as a power source, a fluid source, or a vacuum source, as needed. In some embodiments, the one or more operational sources supplied to the end effector 408 can be included or stored in the base 410.

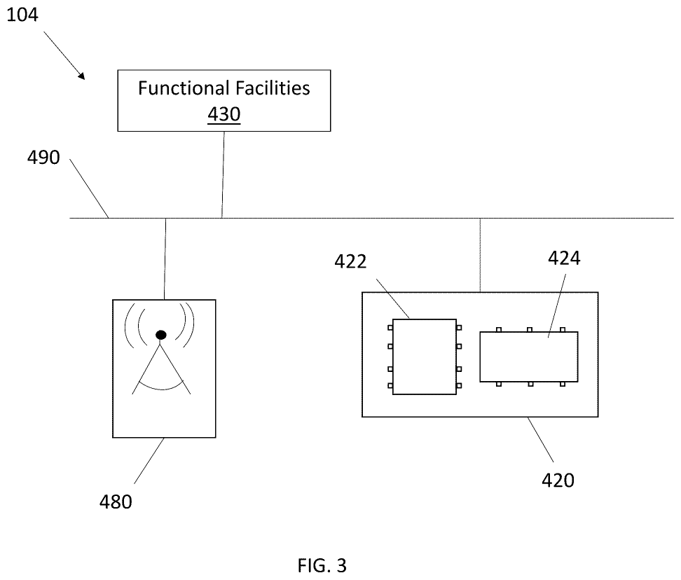

Referring now to FIG. 3, internal hardware components of the robotic cart 104. according to some embodiments, are depicted. The robotic cart 104 can include control circuitry 420 including a processor 422 and a memory module 424. In some embodiments, the control circuitry 420 can be an electronic control unit. In some embodiments, the robotic cart 104 can include a communications module 480). In some embodiments, the robotic cart 104 can include one or more other functional facilities 430. The control circuitry 420 and the various functional facilities 430 can be communicatively coupled to one another via a bus 490. The robotic cart 104 can be coupled to a power supply for supplying power to the control circuitry 420 and various functional facilities 430.

The processor 422 can include any processing component(s) configured to receive and execute instructions. In some embodiments, the instructions can be in the form of one or more processor-readable instructions or instruction sets stored in the memory module 424. In some embodiments, the processor 422 can be an electric controller, an integrated circuit, a microchip, a computer, or any other computing device. In some embodiments, the processor 422 is communicatively coupled to the other functional facilities 430 of the robotic cart 104 via the bus 490. In some embodiments, the bus 490 can communicatively couple any number of processors 422 with one another, and allow the components and functional facilities 430 coupled to the bus 490 to operate in a distributed computing environment. In some embodiments, each functional facility 430 or component of the robotic cart 104 can operate as a node that can send and/or receive data. In some embodiments, the robotic cart 104 can include more than one processor 422.

As noted above, in some embodiments, the control circuitry 420 includes the memory module 424. The memory module 424 can be communicatively coupled to the one or more processors 422. In some embodiments, the memory module 424 can include RAM, ROM, flash memories, hard drives, or any device capable of storing processor-readable instructions such that the processor-readable instructions can be accessed and executed by the one or more processors 422. The processor-readable instructions can include logic or algorithm(s) written in any programming language of any generation (e.g., 1GL, 2GL, 3GL, 4GL, or 5GL) such as, for example, machine language that can be directly executed by the processor 422, or assembly language, object-oriented programming (OOP), scripting languages, microcode, and the like, that can be compiled or assembled into processor-readable instructions and stored on the memory module 424. In some embodiments, the processor-readable instructions can be written in a hardware description language (HDL), such as logic implemented via either a field-programmable gate array (FPGA) configuration or an application-specific integrated circuit (ASIC), or their equivalents. Accordingly, the methods described herein can be implemented in any conventional computer programming language, as pre-programmed hardware elements, or as a combination of hardware and software components.

In some embodiments, the bus 490 can be formed from any medium that is capable of transmitting a signal such as, for example, conductive wires. conductive traces, optical waveguides, or the like. In some embodiments, the bus 490 can be formed from a combination of mediums capable of transmitting signals. The bus 490 communicatively couples the various components and functional facilities 430 of the robotic cart 104, such as those depicted in FIG. 3. As used herein, the term “communicatively coupled” means that coupled components are capable of exchanging data signals with one another such as, for example, electrical signals via conductive medium, electromagnetic signals via air, optical signals via optical waveguides, and the like.

The one or more other functional facilities 430 can each include one or more hardware components and/or one or more software components for the robotic cart 104 to execute one or more instructions or processes. For instance, in some embodiments, the memory module 424 can include instructions executable by the processor 422, and upon executing the instructions, the processor 422 can instruct the one or more functional facilities 430 to perform one or more tasks or functionalities described below.

In some embodiments, the robotic cart 104 can include a driver functional facility. For instance, in some embodiments, the driver functional facility can be communicatively coupled to one or more motors to drive or move the robotic arm 402. The one or more motors can be configured to drive or move one or more segments (e.g., 402A-C) of the robotic arm 402. For instance, the one or more motors can be configured to articulate each robotic arm segment 402A-C independently of each other. In some embodiments, the one or more motors can drive each segment 402A-C of the robotic arm to achieve a desired axial motion, angular motion, or rotation of each segment 402A-C. In some embodiments, the driver functional facility can selectively position the end effector 408 in a surgical site.

In some embodiments, the robotic cart 104 can include an actuation functional facility. In some embodiments, the actuation functional facility can be configured to control actuation of the end effector 408. For instance, the robotic cart 104 can include one or more valves, pistons, levers, triggers, and the like configured to selectively actuate the end effector 408. For instance, in some embodiments, the actuation functional facility can be configured to actuate (e.g., fire) a laser of the end effector 408, to actuate one or more movable blades of the end effector 408, to provide a supply of fluid to and out of the end effector 408, to actuate a suction element of the end effector 408, and the like. In some embodiments, a separate system that interacts with the robot cart 104 and/or the end effector 408 can control actuation of the end effector 408.

In some embodiments, the robotic cart 104 can include an imaging functional facility. In some embodiments, the imaging functional facility can be configured to acquire imaging data (e.g., video or still images) on the surgical site and the operating environment of the end effector 408 (e.g., in the surgical site). In some embodiments, the imaging functional facility gathers the imaging data from one or more imaging sensors. In some embodiments, the imaging sensors can be included in the end effector 408. For instance, in some embodiments, the adapter 406 can receive one or more end effectors 408 at a time, and one of the one or more end effectors 408 is an imaging device, such as a cystoscope, including the one or more imaging sensors. In some embodiments. the imaging device or imaging sensors can be integrated with a dual-purpose end effector 408. For instance, a resectoscope can include an internal lumen for receiving a cystoscope or the imaging device or sensor, and for positioning the imaging sensors in the operating environment of the resectoscope (e.g., in the surgical site). In some embodiments, the imaging functional facility is configured to generate real-time imaging data. A “frame” of image data, as used herein, refers to a set of image data collected by the module at a fixed point in time. A frame of image data can be a still image, or a “slice” of video sensor data at a certain point in time. That is, video data can be considered a collection of frames of imaging data over time.

In some embodiments, the robotic cart 104 can include a locating functional facility. In some embodiments, the locating functional facility can be configured to determine or detect the location (e.g., at least one of the position or the orientation) of the end effector 408. In some embodiments, the functional facility can be communicatively coupled to one or more location sensors positioned on the robotic arm 402 or adapter 406. In some embodiments, the functional facility can be configured to collect inverse kinematic data on the robotic arm 402 or data on the state of the one or more motors for moving the robotic arm 402. In such embodiments, the reverse kinematic data or motor state data can be used to determine a location of the end effector 408. In some embodiments, the locating functional facility can be configured to determine or detect a direction and magnitude of force acting on the end effector 408. In some embodiments, the locating functional facility can be communicative coupled to one or more force or torque sensors on the robotic arm 402 that can detect a force acting on the end effector 408 coupled to the robotic arm 402.

In some embodiments, the communications module 480) can be communicatively coupled to the controller 422 via the bus 490. The communications module 480) can include one or more hardware components capable of transmitting or receiving data with external devices or servers directly or via a network, such as an external network. Accordingly, the communications module 480 can include a communication transceiver for sending or receiving any wired or wireless communication. For example, the communications module 480 can include an antenna, a modem, LAN port, Wi-Fi card, WiMax card, mobile communications hardware, near-field communication hardware, satellite communication hardware and/or any wired or wireless hardware for communicating with other networks or devices. In some embodiments, the communications module 480) can include hardware configured to operate in accordance with the Bluetooth wireless communication protocol and can include a Bluetooth send/receive module for sending and receiving Bluetooth communications.

In some embodiments, the robotic cart 104 can be communicatively coupled to a network, such as an external network. In some embodiments, the external network can include one or more computer networks (e.g., a cloud network, a personal area network, a local area network, grid computing network, wide area network, and the like), cellular networks, satellite networks, or combinations thereof. Accordingly, the robotic cart 104 can be communicatively coupled to the external network via wires, via a wide area network, via a local area network, via a personal area network, via a cellular network, via a satellite network, or the like. Suitable local area networks can include wired Ethernet and/or wireless technologies such as, for example, wireless fidelity (Wi-Fi). Suitable personal area networks can include wireless technologies such as, for example, IrDA, Bluetooth, Wireless USB, Z-Wave, ZigBee, and/or other near field communication protocols. Suitable personal area networks can similarly include wired computer buses such as, for example, USB and FireWire. Suitable cellular networks can include, but are not limited to, technologies such as LTE, WiMAX, UMTS, CDMA, and GSM.

In some embodiments, the communications module 480 can communicatively couple the robotic cart 104 with the console cart 102, as further discussed below. In some embodiments, the communications module 480 can enable the transmission of data and other information on the operation of the robot cart 104 to the console cart 102.

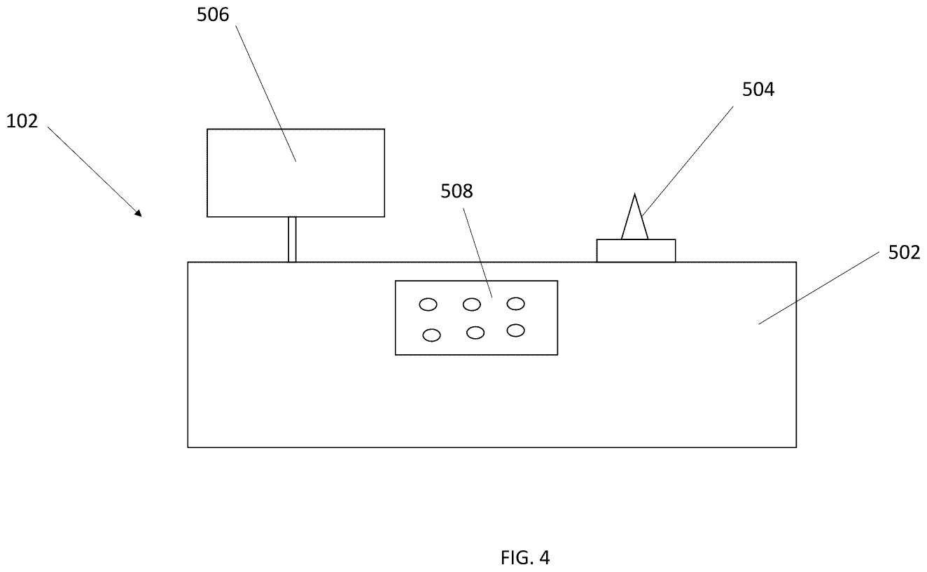

Referring now to FIG. 4, the console cart 102 is depicted according to some embodiments. In some embodiments, the console cart 102 can include a platform 502. In some embodiments, the platform 502 can include one or more wheels such that the console cart 102 is easily movable to a desired location in an operating room, hospital, or other setting. The console cart 102 can be positioned remotely from the robotic cart 104.

In some embodiments, the console cart can include an input device 504. The input device 504 can be a joystick, computer mouse, hand-held controller, a touch screen, or any other device for receiving input from the surgeon 200. In some embodiments, the input device 504 can be manipulated by the surgeon 200 to impart motion to the input device 504 or one or more components thereof. In some embodiments, actuation of the input device 504 can drive motion of the robotic arm 402.

In some embodiments, the console cart 102 can include a display 506. The display 506 can display a user interface. The user interface can be an input/output device that presents information to the surgeon 200 and can receive inputs or commands from the surgeon 200. For instance, in some embodiments, the display 506 can be a touch screen or other display that presents one or more icons, drop down menus, fillable text boxes, buttons, and the like that the surgeon 200 can actuate to provide an input. In some embodiments, the user interface of the display 506 can be manipulated by the surgeon (e.g., the surgeon can interact with the touch screen) to drive motion of the robotic arm 402 (e.g., to position the end effector 408). In some embodiments, the input can be information to be processed by the console cart 102 or a command to be executed by the robotic cart 104. As discussed in greater detail below, the user display 506 can display imaging data generated by the robotic cart 104, such as real-time video of the surgical site around the end effector 408. As discussed in greater detail below, the display 506 can display one or more maps of the surgical site. As discussed in greater detail below, the display 506 can display one or more visuals containing processed data from video and robot telemetry.

In some embodiments, the console cart 102 can include a control panel 508. In some embodiments, the control panel 508 can be used to adjust one or more settings or actuate one or more components related to the console cart 102 or the robotic cart 104. In some embodiments, the control panel 508 can include one or more buttons, switches, dials, pedals, or other actuators. It should be appreciated that the functionalities of the control panel 508 can, in some embodiments, be performed by the user interface of the display 506.

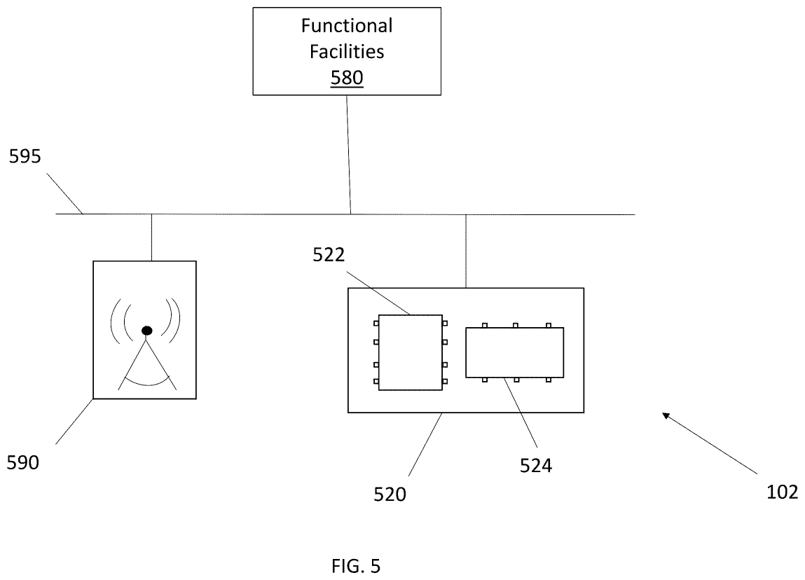

Referring now to FIG. 5, internal hardware components of the console cart 102, according to some embodiments, are depicted. The console cart 102 can include control circuitry 520 including a processor 522 and a memory module 524. In some embodiments, the control circuitry 520) can be an electronic control unit. In some embodiments, the console cart 102 can include a communications module 590. In some embodiments, the console cart 102 include one or more functional facilities 580. The control circuitry 520 and the various functional facilities 580 can be communicatively coupled to one another via a bus 595. The console cart 102 can be coupled to a power supply for supplying power to the control circuitry 520) and various functional facilities 580. The one or more functional facilities 580 can each include one or more hardware components and/or one or more software components for the console cart 102 to execute one or more functionalities or processes. For instance, in some embodiments, the memory module 525 can include instructions executable by the processor 522, and upon executing the instructions, the processor 522 can instruct the one or more functional facilities 580 to perform one or more tasks or functionalities described below.

The processor 522 can include any processing component(s) configured to receive and execute instructions. In some embodiments, the instructions can be in the form of one or more processor-readable instructions or instruction sets stored in the memory module 524. In some embodiments, the processor 522 can be an electric controller, an integrated circuit, a microchip, a computer, or any other computing device. In some embodiments, the processor 522 is communicatively coupled to the one or more functional facilities 580 of the console cart 102 via the bus 595. In some embodiments, the bus 595 can communicatively couple any number of processors 522 with one another and allow the components and functional facilities 580 coupled to the bus 595 to operate in a distributed computing environment. In some embodiments, each module or component of the console cart 102 can operate as a node that can send and/or receive data. In some embodiments, the console cart 102 can include more than one processor 522.

As noted above, in some embodiments, the control circuitry 520 includes the memory module 524. The memory module 524 can be communicatively coupled to the one or more processors 522. In some embodiments, the memory module 524 can include RAM, ROM, flash memories, hard drives, or any device capable of storing processor-readable instructions such that the processor-readable instructions can be accessed and executed by the one or more processors 522. The processor-readable instructions can include logic or algorithm(s) written in any programming language of any generation (e.g., 1GL, 2GL, 3GL, 4GL, or 5GL) such as, for example, machine language that can be directly executed by the processor 522, or assembly language, object-oriented programming (OOP), scripting languages, microcode, and the like, that can be compiled or assembled into processor-readable instructions and stored on the memory module 524. In some embodiments, the processor-readable instructions can be written in a hardware description language (HDL), such as logic implemented via either a field-programmable gate array (FPGA) configuration or an application-specific integrated circuit (ASIC), or their equivalents. Accordingly, the methods described herein can be implemented in any conventional computer programming language, as pre-programmed hardware elements, or as a combination of hardware and software components.

In some embodiments, the bus 595 can be formed from any medium that is capable of transmitting a signal such as, for example, conductive wires, conductive traces, optical waveguides, or the like. In some embodiments, the bus 595 can be formed from a combination of mediums capable of transmitting signals. The bus 595 communicatively couples the various components and functional facilities 580 of the console cart 102, such as those depicted in FIG. 5.

In some embodiments, the communications module 590 can be communicatively coupled to the controller 522 via the bus 595. The communications module 590 can include one or more hardware components capable of transmitting or receiving data with external devices or servers directly or via a network, such as an external network. Accordingly, the communications module 590) can include a communication transceiver for sending or receiving any wired or wireless communication. For example, the communications module 590) can include an antenna, a modem, LAN port, Wi-Fi card, WiMax card, mobile communications hardware, near-field communication hardware. satellite communication hardware and/or any wired or wireless hardware for communicating with other networks or devices. In some embodiments, the communications module 590 can include hardware configured to operate in accordance with the Bluetooth wireless communication protocol and can include a Bluetooth send/receive module for sending and receiving Bluetooth communications.

In some embodiments, the console cart 102 can be communicatively coupled to a network, such as an external network. In some embodiments, the external network can include one or more computer networks (e.g., a cloud network, a personal area network, a local area network, grid computing network, wide area network, and the like), cellular networks, satellite networks, or combinations thereof. Accordingly, the console cart 102 can be communicatively coupled to the external network via wires, via a wide area network, via a local area network, via a personal area network, via a cellular network, via a satellite network, or the like. Suitable local area networks can include wired Ethernet and/or wireless technologies such as, for example, wireless fidelity (Wi-Fi). Suitable personal area networks can include wireless technologies such as, for example, IrDA, Bluetooth, Wireless USB, Z-Wave, ZigBee, and/or other near field communication protocols. Suitable personal area networks can similarly include wired computer buses such as, for example, USB and FireWire. Suitable cellular networks can include, but are not limited to, technologies such as LTE, WiMAX, UMTS, CDMA, and GSM.

In some embodiments, the communications module 590 can communicatively couple the console cart 102 with the robotic cart 104, as further discussed below. In some embodiments, the communications module 590) can enable the reception of data and other information on the operation of the robot cart 104 generated by the robot cart 104, as discussed above, at the console cart 102, as discussed in greater detail below. In some embodiments, the communications module 590 can enable the transmission of instructions from the console cart 102 to the robotic cart 104, which can then be executed by the robot cart 104, as discussed in greater detail below.

In some embodiments, the console cart 102 is configured to receive the imaging data generated by the robotic cart 104. In some embodiments, the console cart 102 includes a landmark functional facility. In some embodiments, the landmark functional facility can be configured to identify landmarks including, but not limited to, the Verumontanum, the bladder neck, the ureteric orifices, the external urethral sphincter, the bladder wall, ejaculatory ducts, bladder neck fibers, prostatic blood vessels, prostatic capsule, stones, tumors, and diverticuli in the imaging data. In some embodiments, the landmark functional facility can identify boundaries between different tissue types in the imaging data. As an example, the landmark functional facility can identify a boundary between the prostatic capsule and the prostate tissue contained within (which in the case of HoLEP can be enlarged tissue (e.g., an adenoma)).

In some embodiments, the console cart 102 is configured to receive the end effector 408 location data generated by the robotic cart 104. In some embodiments, the console cart 102 include a relative locating functional facility. In some embodiments, the relative locating functional facility can be configured to receive the end effector 408 location data from the robotic cart 104 and determine the relative location (e.g., at least one of position or orientation) of the end effector 408 with respect to the tissue (including anatomical landmarks or tissue boundaries) in the imaging data. In some embodiments, relative locating functional facility can be configured to determine the depth of the tissue in front of or surrounding the end effector 408 (e.g., the distance between the end effector 408 and the tissue in front of the end effector 408). In some embodiments, based on the end effector 408 location data, the relative locating functional facility can determine the depth of tissue in front of the end effector 408 in the 2D imaging data received from the robotic cart 104. In some embodiments, the relative locating functional facility can determine the depth of tissue in front of the end effector 408 based on the imaging data.

In some embodiments, the console cart 102 can include a mapping functional facility. In some embodiments, the mapping functional facility can generate a map of the surgical site. The map of the surgical site can be a graphical representation of the surgical site outside of the field of view represented in the imaging data received from the robotic cart 104 with the end effector 408 at its current location. That is, the map can be a projection of an anatomy of the surgical site surrounding the real-time imaging data. In some embodiments, the map of the surgical site can be a graphical representation of all locations the end effector 408 has been from a start of a procedure (e.g., based on all previously gathered imaging data during the procedure). In some embodiments, the map can be generated based on imaging data previously received from the robot cart 104 (e.g., imaging data gathered at previous locations of the end effector 408). In some embodiments, the map can show the contours, shape, size, location, etc. of the tissue in the surgical site.

In some embodiments, the console cart 102 can include a display functional facility. In some embodiments, the display functional facility can be configured to display the map of the surgical site and the current imaging data received from the end effector 408 together on the display 506. In some embodiments, the current imaging data received from the end effector 408 at its current location can be displayed over the map of the surgical site. In some embodiments, the current imaging data received from the end effector 408 at its current location can be relatively positioned on the map of the surgical site at the current location of the end effector 408. In some embodiments, the map of the surgical site can include a point cloud. The point cloud can generally comprise a discrete set of data points, each having a set of Cartesian coordinates, which together create a three-dimensional representation of a surface (e.g., showing its depth, contours, etc.). For instance, by tracking the depth of various tissue points in front of the end effector 408 or imaging sensors as they are moved through the surgical site, a point cloud showing the contours and shape of the tissue in the surgical site can be developed.

In some embodiments, the console cart 102 can include an input functional facility. In some embodiments, the input functional facility can receive input from the surgeon to move the end effector 408 and provide an instruction to the robotic cart 104 to move the end effector 408 based on the input. In some embodiments, the input functional facility can receive the input from the surgeon on the display 506. In some embodiments, the input can be in the form of the user interacting with a touch screen of the display 506. For instance, the surgeon can swipe his or her finger on the display 506 to move the end effector 408. The console cart 102 can instruct the robot cart 104 to move the end effector 408 at different speeds depending on where or how the user interacts with the display 506, as discussed in further detail below. As a non-limiting example, the surgeon swiping his finger in the map shown on the display 506 (“dragging” the map) can result in an instruction to move the end effector 408 in the direction indicated by the input at a first speed. As a non-limiting example, the surgeon “dragging” a marker for the imaging data generated at the current position or location of the end effector can result in an instruction to move the end effector 408 in the direction indicated by the input at a second speed. In some embodiments, the input functional facility and the console cart 102 can generate the instruction to move the end effector 408 freely in three directions in accordance with the input received from the surgeon.

In some embodiments, the input functional facility and the console cart 102 can generate the instruction to move the end effector 408 freely in two directions in accordance with the input received from the surgeon. In some embodiments, the console cart 102 can include a depth movement functional facility. In some embodiments, the depth movement functional facility can autonomously determine the instruction to move the end effector in the third direction. In some embodiments, the first and second directions instructed by the surgeon can be parallel to the plane of tissue in front of the end effector 408. In some embodiments, the third direction can be perpendicular to the plane of the tissue in front of the end effector 408. In some embodiments, the depth movement functional facility can generate the instruction to move the end effector 408 such that the end effector 408 remains a desired distance from the tissue in front of the end effector 408 (e.g., in the third direction). In some embodiments, the desired distance can be set by a user.

It should be appreciated that the configuration of the system 100 discussed above is merely one non-limiting example of the layout of the system 100. That is, in some embodiments, the system 100 can include a single console that includes all of the above-described features of the robotic cart 104 and the console cart 102. That is, data collection, analysis, presentation, and the like, do not need to be distributed between different consoles. Instead, such determinations can be made locally on a single console that includes the hardware and software of the both the console cart 102 and the robotic cart 104. That is to say, the system 100 can include control circuitry, including at least one processing component and at least one memory component, and any number of the above functional facilities communicatively coupled to the control circuitry, in any desired layout or architecture for performing the operations described herein.

Moreover, it should be appreciated that the above-discussed functional facilities can be distributed in other manners than those presented above. For instance, and merely as an example, the robotic cart 104 may include the landmark functional facility and then transmit its generated data on landmark and tissue boundary identification to the console cart 102. Indeed, in some embodiments, any or all of the analyses can be performed on the robotic cart 104 and transmitted to the console cart 102, which can function merely to present such information via the display functional facility or and to receive user inputs via the input functional facility.

Moreover, it should be appreciated that the “console cart” 102 need not include the hardware elements discussed with respect to FIG. 4. Instead, the hardware of the console cart 102 discussed herein is merely one non-limiting example. The functionality of the discussed “console cart” 102 can be entirely housed within any processing device. For instance, the “console cart” 102 can merely be a hand-held device that can process data, communicate with the robot cart 104, present information to the surgeon, and receive surgeon input (on a touch screen for instance). In some embodiments, the “console cart” 102 can be a standalone computer, a dedicated electronic device, a tablet, a smartphone, etc.

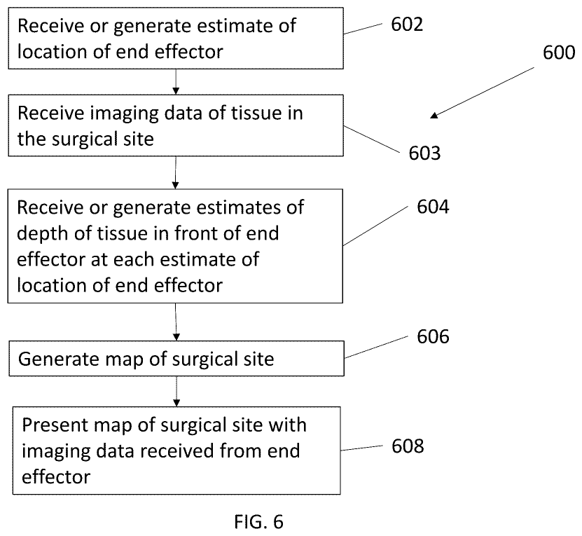

Referring now to FIG. 6, an example method 600 of presenting a map of the surgical site is depicted. The map and imaging data can be presented on the display 506. In a step 602 the system 100 can receive or generate an estimate of the location of the end effector 408. For instance, in a step 602, the locating functional facility can receive or generate an estimate of the location of the end effector 408. In particular, the system 100 can receive or generate an estimate of the location of the distal end of the end effector 408. For instance, the locating functional facility can receive or generate an estimate of the location of the distal end of the end effector 408. In a step 603, the system 100 can receive or generate imaging data of the surgical site. For instance, in some embodiments, the imaging functional facility can receive or generate imaging data of the surgical site. The imaging data can contain images of tissue in the surgical site. In some embodiments, the imaging data is real-time imaging data gathered of the surgical site. In some embodiments, the method 600 can include a step 604. In step 604, the system 100 can receive or generate an estimate of the depth of tissue in front of the end effector 408 (e.g., the distance from the end effector 408 to the tissue in front of the end effector 408) at each estimate of the location of the end effector 408. For instance, in step 604, the locating functional facility can receive or generate an estimate of the depth of tissue in front of the end effector 408 (e.g., the distance from the end effector 408 to the tissue in front of the end effector 408) at each estimate of the location of the end effector 408.

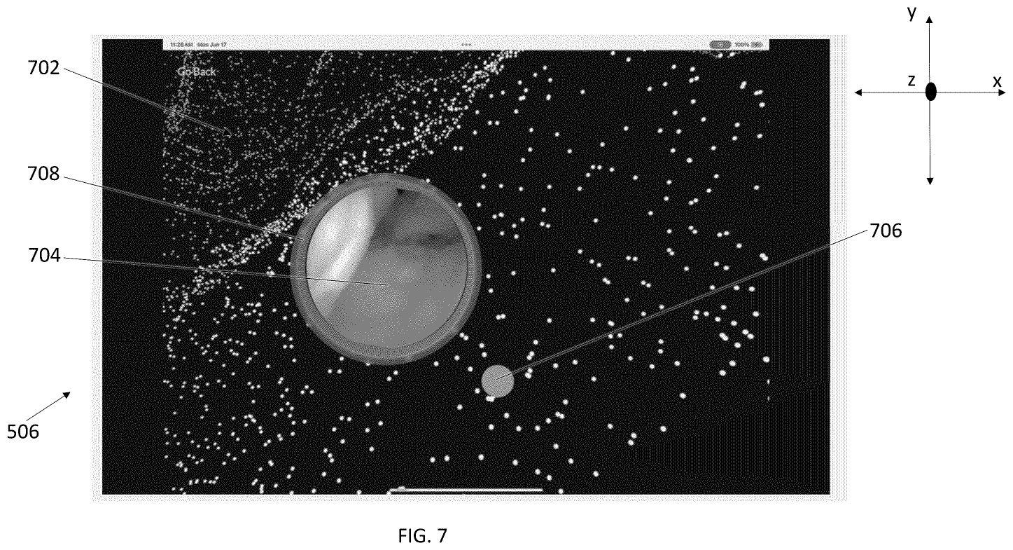

In step 606, the system 100 can generate a map of the surgical site. For instance, the mapping functional facility can generate a map of the surgical site. In some embodiments, the map of the surgical site can be based on the prior received location estimates and prior received depth estimates received in steps 602 and 604. In some embodiments, the map of the surgical site can be based on prior received real-time imaging data from the robotic cart 104. The map of the surgical site can be a graphical representation of the surgical site beyond the field-of-view presented in the imaging data currently received from the robotic cart 104. For instance, referring to FIG. 7, the map 702 of the surgical site can be a point cloud or other graphical representation that generally shows the contours, shape, size, position, etc. of the tissue in the surgical site, and particularly the contours, size, position etc. of tissue in the surgical site surrounding the tissue currently displayed in imaging data 704. The graphical representation can be such that a surgeon can perceive the three-dimensionality of the surgical site on the two-dimensional display 506.

In step 608, the system 100 can present the map of the surgical site with the imaging data currently received from the robotic cart 104 or end effector 408. For instance, the display functional facility can present the map of the surgical site with the imaging data currently received of the surgical site. Referring again to FIG. 7, the imaging data 704 is shown. The map 702 of the surgical site can be seen to extend beyond the boundaries of the imaging data 704. Therefore, the system 100 provides the surgeon with more information to understand the position of the end effector 408 in the anatomy of the surgical site. The imaging data 704 is presented at a location on the map 702 that corresponds to the location of the end effector 408 in the surgical site (e.g., the location in the map 702 that the imaging data 704 is currently being generated at by the end effector 408).

In some embodiments, as new data is gathered at steps 602, 603, and 604 of the method 600, the map 702 can be constantly updated by the system 100. For instance, as new data is gathered at steps 602, 603, and 604 of the method 600, the map 702 can be constantly updated by the mapping functional facility, and the display can be constantly updated by the display functional facility. In some embodiments, the system 100 presents the imaging data 704 in a border 708. In some embodiments, the display functional facility can present the imaging data 704 in the border 708. The border 708 can present visual cues of forces that are detected on the end effector 408 by one or more sensors of the end effector 408 or the robotic cart 104. The forces can be forces from tissue in the surgical site acting on the end effector 408 (and vice versa). The border can be white when no or neutral force is acting on the end effector 408. In some embodiments, the border can change colors in the direction in which forces are acting on the end effector 408. In some embodiments, the color, shade, brightness, intensity. etc. of the color on the border can indicate the magnitude of the force acting on the end effector 408. In some embodiments, the system 100 can label one or more anatomical landmarks in the map 702 or in the imaging data 704. For instance, the display functional facility can generate a label for the one or more anatomical landmarks, identified by the landmark functional facility, in the map 702 or the imaging data 704. In some embodiments, the various steps of the method 600 can completed by the console cart 102 or shared between the console cart 102 and the robotic cart 104.

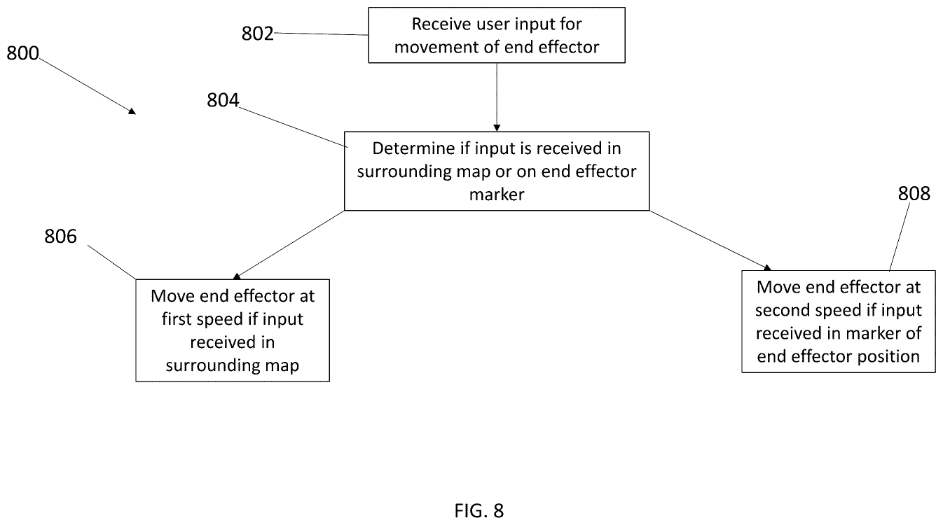

Referring now to FIG. 8, an example method 800 of moving the end effector 408 at variable speeds is depicted. For instance, the driver functional facility can be configured to move the end effector with coarse control (e.g., a faster speed for larger movements) or with fine control (e.g., a slower speed for smaller, more precise movements), based on input received from a user by the input functional facility. In step 802, the system 100 can receive input from the user (e.g., a surgeon) for moving the end effector 408. For instance, the input functional facility can receive input from the user for moving the end effector 408. In some embodiments, the input can be provided via a touch screen on the display 506. In step 804, the system 100 can determine if the input is received in the map 702 or on a marker 706 of the display 506. In some embodiments, the input functional facility can determine if the input is received in the map 702 or on the marker 706. For instance, as shown in step 806, if the user is “scrolling” or “swiping” (e.g. dragging his finger) in the map 702, an instruction can be generated to move the end effector 408 at a first speed and in accordance with the direction of the swiping on the display 506. In some embodiments, the input functional facility can generate an instruction to move the end effector 408 at the first speed. In some embodiments, the driver functional facility can move the end effector 408 at the first speed. The first speed can be for coarse movement.

As shown in step 808, if the surgeon “drags” the marker 706 (e.g., places his finger on the marker 706 and drags his finger on the display 506), an instruction can be generated to move the end effector 408 at a second speed and in accordance with the direction of the finger dragging on the display 506. In some embodiments, the input functional facility can generate an instruction to move the end effector 408 at the second speed. In some embodiments, the driver functional facility can move the end effector 408 at the second speed. The second speed can be for fine movement. The second speed can be less than the first speed. The marker 706 can be presented in a fixed location relative the imaging data 704 (and therefore the position of the end effector 408) shown on the display 506. The marker 706 can be a “handle” for moving the end effector 408 or the view provided by the imaging data 704. The marker 706 obviates the need for the user to place his finger in the imaging data 704 to “drag” the imaging data 704 to move the end effector 408 at the second speed. Therefore, the marker 706 can help the user maintain full visibility of the imaging data 704 being generated of the surgical site. As the end effector 408 moves in response to user input, the display 506, including the map 702 and imaging data 704 is updated accordingly. The ratio of pixels moved on the display 506 to distance moved by the end effector 408 will vary depending on if the system 100 instructs the end effector 408 to be moved at the first speed or the second speed.

In some embodiments, the first speed and the second speed can be set by the user. In some embodiments, one or both of the first speed or the second speed can be variable based on the speed the user moves his finger across the display 506, the direction of movement in the surgical site, or other factors. In some embodiments, one or both of the first speed or the second speed can be variable based on the operational status of the end effector 408 (e.g., whether the end effector is being actuated to perform enucleation, aspirating, cutting, etc.). In some embodiments, the various steps of the method 800 can completed by the console cart 102 or shared between the console cart 102 and the robotic cart 104. For instance. it should be appreciated that the console cart 102 can generate an instruction to move the end effector 408 and the robotic cart 104 can execute the instruction.

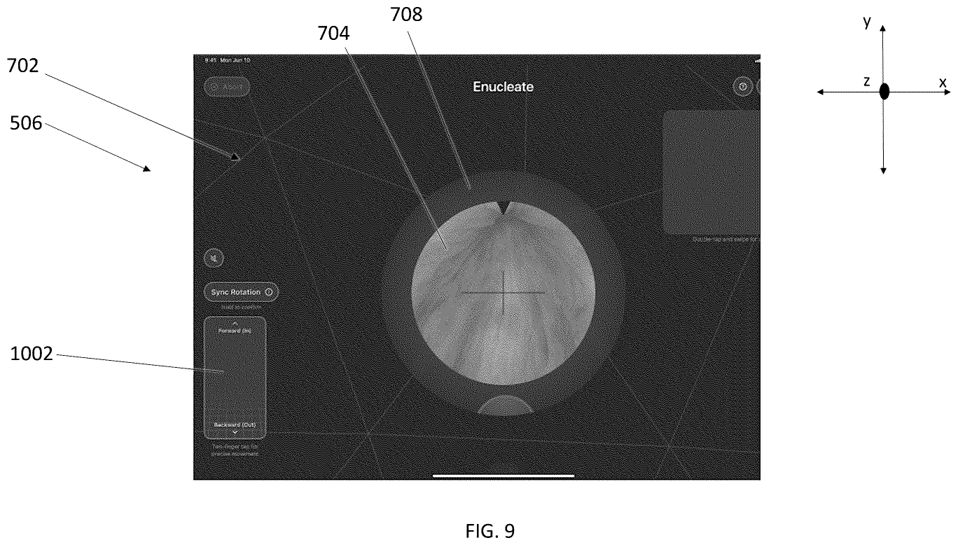

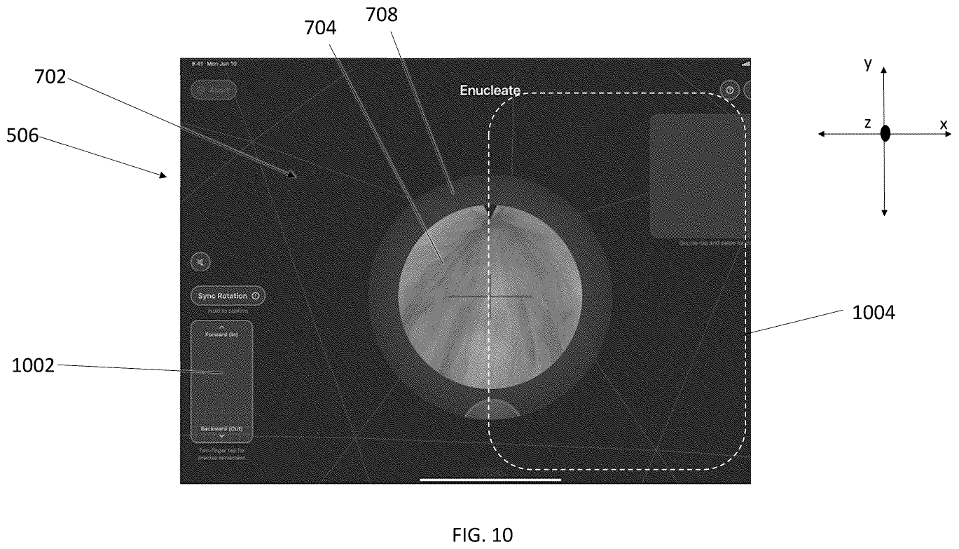

Referring to FIG. 9, another embodiment of a graphical user interface presented on the display 506 is depicted. The user interface can be generated by the display functional facility. The user interface can include the map 702, imaging data 704, and the border 708, as discussed with respect to FIG. 7. The user interface can include a first designated touch area 1002. The first designated touch area 1002 can be overlayed on top of the map 702. Referring to FIG. 10, another embodiment of a graphical user interface presented on the display 506 is depicted. The user interface of FIG. 10 can be generated by the display functional facility. The user interface of FIG. 10 largely resembles of the user interface of FIG. 9, but includes a second designated touch area 1004. The boundary of the second designated touch area 1004 is shown in FIG. 10 in dashed line. In some embodiments, the second designated touch area 1004 can include at least a portion of the imaging data 704. In some embodiments, the second designated touch area 1004 can include at least a portion of the map 702. In some embodiments, the second designated touch area 1004 can include at least a portion of the imaging data 704 and at least a portion of the map. In some embodiments, the second designated touch area 1004 can include at least a portion of the border 708. In some embodiments, the second designated touch area 1004 can include at least a portion of the imaging data 704, at least a portion of the map 702, and at least a portion of the border 708. In some embodiments, the second designated touch area 1004 can be offset a distance from the first designated touch area 1002. In some embodiments, the first designated touch area 1002 is on a first side of the user interface (e.g., left of center of the user interface), and the second designated touch area 1004 is on a second side of the user interface (e.g., right of center of the user interface). This can avoid the user providing accidental input in the first designated touch area 1002 instead of the second designated touch area 1004, and vice versa. It should be appreciated that the above-described arrangement of the first designated touch area 1002 and the second designated touch area 1004 is merely one non-limiting example, and they can be arranged in different manners with respect to each other and the center of the user interface.

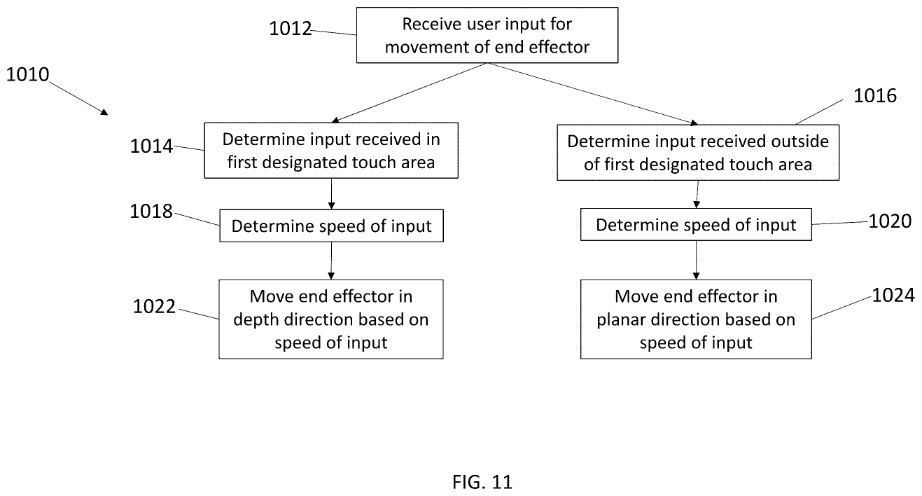

Referring now to FIG. 11, an example method 1010 of moving the end effector 408 at variable speeds is depicted. For instance, the driver functional facility can be configured to move the end effector with coarse control (e.g., a faster speed for larger movements) or with fine control (e.g., a slower speed for smaller, more precise movements), based on input received from a user by the input functional facility. In step 1012, the system 100 can receive input from the user (e.g., a surgeon) for moving the end effector 408. For instance, the input functional facility can receive input from the user for moving the end effector 408. In some embodiments, the input can be provided via a touch screen on the display 506. In some embodiments, the system 100, for instance the input functional facility, can determine where on the display 506 the input was received. In a step 1014 of the method 1010, the system 100, for instance the input functional facility, can determine that the input is received in the first designated touch area 1002. Input received in the first designated touch area 1002 can be for controlling movement of the end effector 408 in the depth direction. The depth direction can be toward or away from the tissue in front of the end effector 408. In some embodiments, the depth direction can be perpendicular to the tissue in front of the end effector 408. The depth direction can be toward or away from the tissue displayed in the imaging data 704. In some embodiments, the depth direction can be perpendicular to the tissue displayed in the imaging data 704. It should be appreciated, that in some embodiments, the tissue that is shown in the imaging data 704 is the tissue in front of the end effector 408. The depth direction can be along the longitudinal axis of the end effector 408. The depth direction can be the direction of the z-axis of the coordinate axes shown in FIG. 9 or FIG. 10. It should be understood that the z-axis of the coordinate axes shown in FIG. 9 and FIG. 10, shown as the black dot in the coordinate axes, is coming into and out of the page. In some embodiments, the input received in the first designated touch area 1002 can be the user swiping (e.g., dragging his finger) in the first designated touch area 1002. In some embodiments, swiping in a first direction can provide instruction to move the end effector 408 toward the tissue in front of the end effector 408 or in the imaging data 704, and swiping in a second direction (e.g., opposite the first direction) can provide instruction to move the end effector 408 away from the tissue in front of the end effector 408 or in the imaging data 704. In some embodiments, input received in the first designated touch area 1002 can have no effect on movement of the end effector in the planar direction, discussed further below.

In some embodiments, in step 1018, the system 100, and for instance the input functional facility, can determine the speed of the input received in the first designated touch area 1002. The speed of the input can be, for instance, the speed with which the user drags his finger on the touch screen in the first designated touch area 1002. In some embodiments, in step 1022, the system 100 can move the end effector 408 in the depth direction based on the speed of the input received in the first designated touch area 1002. In some embodiments, the input functional facility can provide instruction to move the end effector 408 in the depth direction based on the speed of the input received in the first designated touch area 1002. In some embodiments, the driver functional facility can move the end effector 408 in the depth direction based on the speed of the input received in the first designated touch area 1002. In some embodiments, the input speed determined in step 1018 can be applied to a non-linear acceleration curve. The non-linear acceleration curve can be, in a non-limiting example, an exponential curve, with the x-axis of the curve being the input speed determined in step 1018 and the y-axis of the curve being the speed with which the end effector 408 is moved in the depth direction. A slow input (e.g., a slow finger swipe) registers on the shallow end of the non-linear acceleration curve, therefore resulting in slower movement of the end effector 408 (e.g., fine control). A fast input (e.g., a fast finger swipe) registers on the steep end of the non-linear acceleration curve, therefore resulting in faster movement of the end effector 408 (e.g., coarse control).

In a step 1016 of the method 1010, the system 100, for instance the input functional facility, can determine that the input is received outside the first designated touch area 1002. Input received outside the first designated touch area 1002 can be for controlling movement of the end effector 408 in the planar direction. The planar direction can generally be in the plane perpendicular to the depth direction. The planar direction can be in the plane parallel to the tissue in front of the end effector 408. The planar direction can in the plane parallel to the tissue displayed in the imaging data 704. It should be appreciated, that in some embodiments, the tissue that is shown in the imaging data 704 is the tissue in front of the end effector 408. The planar direction can be in the plane parallel to the longitudinal axis of the end effector 408. The planar direction can be the x-y plane of the coordinate axes shown in FIG. 9 or FIG. 10. Movement in the planar direction, can involve moving the end effector 408 up, down, or side to side in the surgical site without moving the end effector 408 toward or away from the tissue in front of the end effector 408, for instance.

In some embodiments, the input received outside the first designated touch area 1002 can be the user swiping (e.g., dragging his finger) in the imaging data 704. In some embodiments, the input received outside the first designated touch area 1002 can be the user swiping (e.g., dragging his finger) in the map 702. In some embodiments, the input received outside the first designated touch area 1002 can be the user swiping (e.g., dragging his finger) in the border 708. In some embodiments, the input received outside the first designated touch area 1002 can be the user swiping (e.g., dragging his finger) in two or more of the imaging data 704, the map 702, or the border 708. For instance, in some embodiments, the user can begin a swiping motion in the imaging data 704 and continue the swiping motion through the border 708 and into the map 702. In some embodiments, for the system 100, particularly for instance the input functional facility, to recognize an instruction for planar movement of the end effector 408, the input must be input by the user in the second designated touch area 1004 (e.g., dragging his finger in the second designated touch area 1004). In some embodiments, providing an input on the user interface outside the first designated touch area 1002 and the second designated touch area 1004 is not registered as an instruction for movement of the end effector 408. In some embodiments. depending on the direction of swiping in the x-y plane of the coordinate axes of FIG. 9 and FIG. 10, an instruction can be provided for moving the end effector up/down (e.g., in the direction of the y-axis of the coordinate axes of FIG. 9 and FIG. 10), left/right (e.g., in the direction of the x-axis of the coordinate axes of FIG. 9 and FIG. 10), or both up/down and left/right. In some embodiments, input received outside the first designated touch area 1002 can have no effect on movement of the end effector 408 in the depth direction.

In some embodiments, in step 1020, the system 100, and for instance the input functional facility, can determine the speed of the input received outside the first designated touch area 1002. The speed of the input can be, for instance, the speed with which the user drags his finger on the touch screen outside the first designated touch area 1002. In some embodiments, in step 1024, the system 100 can move the end effector 408 in the planar direction based on the speed of the input received outside the first designated touch area 1002. In some embodiments, the input functional facility can provide instruction to move the end effector 408 in the planar direction based on the speed of the input received outside the first designated touch area 1002. In some embodiments, the driver functional facility can move the end effector 408 in the planar direction based on the speed of the input received outside the first designated touch area 1002. In some embodiments, the input speed determined in step 1020 can be applied to a non-linear acceleration curve. The non-linear acceleration curve can be, in a non-limiting example, an exponential curve, with the x-axis of the curve being the input speed determined in step 1020 and the y-axis of the curve being the speed with which the end effector 408 is moved in the planar direction. A slow input (e.g., a slow finger swipe) registers on the shallow end of the non-linear acceleration curve, therefore resulting in slower movement of the end effector 408 (e.g., fine control). A fast input (e.g., a fast finger swipe) registers on the steep end of the non-linear acceleration curve, therefore resulting in faster movement of the end effector 408 (e.g., coarse control).

In some embodiments, a slow finger swipe in the first designated touch area 1002 can result in movement of the end effector 408 in the depth direction at a slow speed. In some embodiments, a fast finger swipe in the first designated touch area 1002 can result in movement of the end effector 408 in the depth direction at a fast speed. In some embodiments, a slow finger swipe in one or more of the map 702, imaging data 704, or border 708 can result in movement of the end effector 408 in the planar direction at a slow speed. In some embodiments, a fast finger swipe in one or more of the map 702, imaging data 704, or border 708 can result in movement of the end effector 408 in the planar direction at a fast speed.

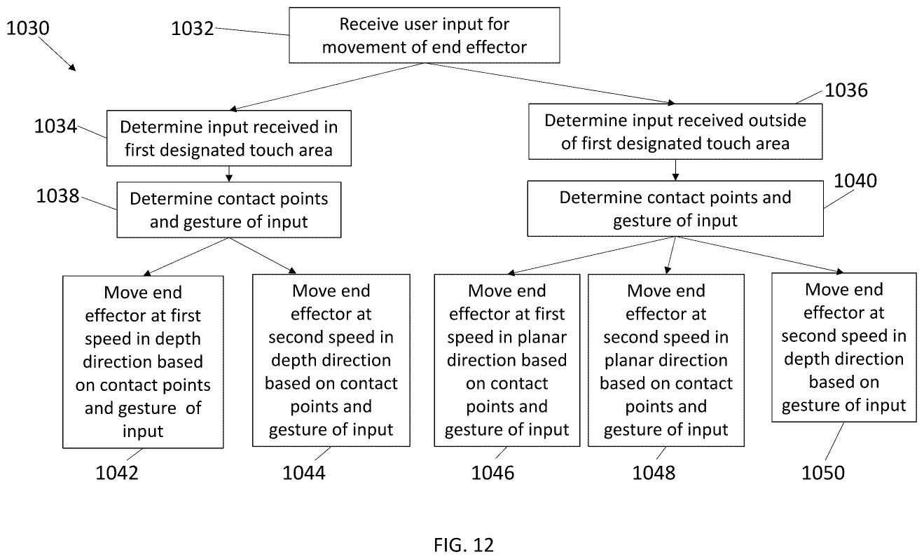

Referring now to FIG. 12, an example method 1030 of moving the end effector 408 at variable speeds is depicted. For instance, the driver functional facility can be configured to move the end effector with coarse control (e.g., a faster speed for larger movements) or with fine control (e.g., a slower speed for smaller, more precise movements), based on input received from a user by the input functional facility. In step 1032, the system 100 can receive input from the user (e.g., a surgeon) for moving the end effector 408. For instance, the input functional facility can receive input from the user for moving the end effector 408. In some embodiments, the input can be provided via a touch screen on the display 506. In some embodiments, the system 100, for instance the input functional facility, can determine where on the display 506 the input was received. In a step 1034 of the method 1010, the system 100, for instance the input functional facility, can determine that the input is received in the first designated touch area 1002. Input received in the first designated touch area 1002 can be for controlling movement of the end effector 408 in the depth direction. The depth direction can be toward or away from the tissue in front of the end effector 408. In some embodiments, depth direction can be perpendicular to the tissue in front of the end effector 408. The depth direction can be toward or away from the tissue displayed in the imaging data 704. In some embodiments, the depth direction can be perpendicular to the tissue displayed in the imaging data 704. It should be appreciated, that in some embodiments, the tissue that is shown in the imaging data 704 is the tissue in front of the end effector 408. The depth direction can be along the longitudinal axis of the end effector 408. The depth direction can be the direction of the z-axis of the coordinate axes shown in FIG. 9 or FIG. 10. It should be understood that the z-axis of the coordinate axes shown in FIG. 9 and FIG. 10, shown as the black dot in the coordinate axes, is coming into and out of the page. In some embodiments, the input received in the first designated touch area 1002 can be the user swiping (e.g., dragging his finger) in the first designated touch area 1002. In some embodiments, swiping in a first direction can provide instruction to move the end effector 408 toward the tissue in front of the end effector 408 or in the imaging data 704, and swiping in a second direction (e.g., opposite the first direction) can provide instruction to move the end effector 408 away from the tissue in front of the end effector 408 or in the imaging data 704. In some embodiments, input received in the first designated touch area 1002 can have no effect on movement of the end effector in the planar direction.

In some embodiments, in step 1038, the system 100, and for instance the input functional facility, can determine the gesture and contact points of the input received in the first designated touch area 1002. Determining the gesture can include, in some embodiments, determining that the input received in the first designated touch area 1002 is a finger swipe. Determining the contact points can, in some embodiments, include determining the number of contact points of the input received in the first designated touch area 1002. For example, the contact points can be a single contact point when the user drags a single finger on the touch screen in the first designated touch area 1002. As another example, the contact points can be two contact points when the user drags two fingers on the touch screen in the first designated touch area 1002.

In some embodiments, the system 100 can move the end effector 408 in the depth direction when a finger swipe gesture is recognized as the input in the first designated touch area 1002. In some embodiments, the system 100 can move the end effector 408 at different speeds in the depth direction depending on the contact points of the input received from the user. In some embodiments, in step 1042, the system 100 can move the end effector 408 at a first speed in the depth direction based on the contact points of the input received in the first designated touch area 1002. In some embodiments, the first speed can be a speed for coarse movement of the end effector 408. In some embodiments, the system 100 can move the end effector 408 at the first speed in the depth direction when the input received in the first designated touch area 1002 includes a single contact point (e.g., a single-finger swipe). In some embodiments, in step 1044, the system 100 can move the end effector 408 at a second speed in the depth direction based on the contact points of the input received in the first designated touch area 1002. In some embodiments, the second speed can be a speed for fine movement of the end effector 408. In some embodiments, the first speed is greater than the second speed. In some embodiments, the system 100 can move the end effector 408 at the second speed in the depth direction when the input received in the first designated touch area 1002 includes two contact points (e.g., a two-finger swipe). In some embodiments, the input functional facility can provide instruction to move the end effector 408 in the depth direction based on the gesture and contact points of the input received in the first designated touch area 1002. In some embodiments, the driver functional facility can move the end effector 408 in the depth direction based on the gesture and contact points of the input received in the first designated touch area 1002.

In a step 1036 of the method 1030, the system 100, for instance the input functional facility, can determine that the input is received outside the first designated touch area 1002. Input received outside the first designated touch area 1002 can be for controlling movement of the end effector 408 in the planar direction or in the depth direction. The planar direction can generally be in the plane perpendicular to the depth direction. The planar direction can be in the plane parallel to the tissue in front of the end effector 408. The planar direction can in the plane parallel to the tissue displayed in the imaging data 704. It should be appreciated, that in some embodiments, the tissue that is shown in the imaging data 704 is the tissue in front of the end effector 408. The planar direction can be in the plane parallel to the longitudinal axis of the end effector 408. The planar direction can be the x-y plane of the coordinate axes shown in FIG. 9 or FIG. 10. Movement in the planar direction can involve moving the end effector 408 up, down, left, or right in the surgical site without moving the end effector 408 toward or away from the tissue in front of the end effector 408, for instance.

In some embodiments, the input received outside the first designated touch area 1002 can be the user swiping (e.g., dragging his finger) in the imaging data 704. In some embodiments, the input received outside the first designated touch area 1002 can be the user swiping (e.g., dragging his finger) in the map 702. In some embodiments, the input received outside the first designated touch area 1002 can be the user swiping (e.g., dragging his finger) in the border 708. In some embodiments, the input received outside the first designated touch area 1002 can be the user swiping (e.g., dragging his finger) in two or more of the imaging data 704, the map 702, or the border 708. For instance, in some embodiments, the user can begin a swiping motion in the imaging data 704 and continue the swiping motion through the border 708 and into the map 702. In some embodiments, the input received outside the first designated touch area 1002 can be the user pinching (e.g., dragging two fingers toward one another, or dragging two fingers away from one another) in the imaging data 704. In some embodiments, the input received outside the first designated touch area 1002 can be the user pinching (e.g., dragging two fingers toward or away from one another) in the map 702. In some embodiments, the input received outside the first designated touch area 1002 can be the user pinching (e.g., dragging two fingers toward or away from one another) in the border 708. In some embodiments, the input received outside the first designated touch area 1002 can be the user pinching (e.g., dragging two fingers toward or away from one another) in two or more of the imaging data 704, the map 702, or the border 708. For instance, in some embodiments, the user can begin a pinching motion in the map 702 and continue the pinching motion through the border 708 and into the imaging data 704.

In some embodiments, for the system 100, particularly for instance the input functional facility, to recognize an instruction for planar or depth movement of the end effector 408 outside of the first designated touch area 1002, the input must be input by the user in the second designated touch area 1004 (e.g., dragging his finger or pinching his fingers in the second designated touch area 1004). In some embodiments, providing an input on the user interface outside the first designated touch area 1002 and the second designated touch area 1004 is not registered as an instruction for movement of the end effector 408. In some embodiments, depending on the direction of swiping in the x-y plane of the coordinate axes of FIG. 9 and FIG. 10, an instruction can be provided for moving the end effector up/down (e.g., in the direction of the y-axis of the coordinate axes of FIG. 9 and FIG. 10), left/right (e.g., in the direction of the x-axis of the coordinate axes of FIG. 9 and FIG. 10), or both up/down and left/right. In some embodiments, a swiping input received outside the first designated touch area 1002 can have no effect on movement of the end effector 408 in the depth direction. In some embodiments, depending on the direction of pinching gesture of the input (e.g., moving two fingers toward each other or away from each other), an instruction can be provided for moving the end effector 408 in the depth direction toward or away from the tissue in front of the end effector 408 or in the imaging data 704. For instance, a pinching gesture in which two fingers are dragged away from each other can result in moving the end effector toward the tissue in front of the end effector 408 or in the imaging data 704, and a pinching gesture in which two fingers are dragged toward each other can result in moving the end effector away from the tissue in front of the end effector 408 or in the imaging data 704. In some embodiments, a pinching input received outside the first designated touch area 1002 can have no effect on movement of the end effector in the planar direction.

In some embodiments, in step 1038, the system 100, and for instance the input functional facility, can determine the gesture and contact points of the input received outside the first designated touch area 1002. Determining the gesture can include, in some embodiments, determining whether the input received outside the first designated touch area 1002 is a finger swipe or a pinch. For instance, a swipe can be recognized by a single contact point moving across the display (e.g., a single-finger swipe) or multiple contact points moving across the screen substantially in the same directions (e.g., a two-finger swipe). A pinch can be recognized by two contact points moving roughly parallel and in opposite directions to one another (e.g., two fingers being brought to together or moved away from each other). Determining the contact points can, in some embodiments, can include determining the number of contact points of the input received outside the first designated touch area 1002. For example, the contact points can be a single contact point when the user drags a single finger on the touch screen outside the first designated touch area 1002. As another example, the contact points can be two contact points when the user drags two fingers on the touch screen outside the first designated touch area 1002.

In some embodiments, the system 100 can move the end effector 408 in the planar direction when a finger swipe gesture is recognized as the input outside the first designated area 1002. In some embodiments, the system 100 can move the end effector 408 at different speeds in the planar direction depending on the contact points of the input received from the user outside the first designated touch area 1002. In some embodiments, in step 1046, the system 100 can move the end effector 408 at a first speed in the planar direction based on the contact points of the input received outside the first designated touch area 1002. In some embodiments, the first speed can be a speed for coarse movement of the end effector 408. In some embodiments, the system 100 can move the end effector 408 at the first speed in the planar direction when the input received outside the first designated touch area 1002 includes a single contact point (e.g., a single-finger swipe). In some embodiments, in step 1048, the system 100 can move the end effector 408 at a second speed in the planar direction based on the contact points of the input received outside the first designated touch area 1002. In some embodiments, the second speed can be a speed for fine movement of the end effector 408. In some embodiments, the first speed is greater than the second speed. In some embodiments, the system 100 can move the end effector 408 at the second speed in the planar direction when the input received outside the first designated touch area 1002 includes two contact points (e.g., a two-finger swipe). In some embodiments, the input functional facility can provide instruction to move the end effector 408 in the planar direction based on the contact points of the input received outside the first designated touch area 1002. In some embodiments, the driver functional facility can move the end effector 408 in the planar direction based on the contact points of the input received outside the first designated touch area 1002.

In some embodiments, the system 100 can move the end effector 408 in the depth direction when a pinch gesture is recognized as the input outside the first designated area 1002. In some embodiments, in step 1050, the system 100 can move the end effector 408 at the second speed in the depth direction based on the gesture of the input received outside the first designated touch area 1002 (e.g., a pinching gesture received outside the first designated touch area 1002). In some embodiments, the input functional facility can provide instruction to move the end effector 408 in the depth direction based on the gesture of the input received outside the first designated touch area 1002. In some embodiments, the driver functional facility can move the end effector 408 in the depth direction based on the gesture of the input received outside the first designated touch area 1002.

In some embodiments, a single-finger swipe in the first designated touch area 1002 can result in movement of the end effector 408 in the depth direction at a first speed for coarse movement. In some embodiments, a two-finger swipe in the first designated touch area 1002 can result in movement of the end effector 408 in the depth direction at second speed for fine movement, where the first speed is faster than the second speed. In some embodiments, a pinch gesture in one or more of the map 702. imaging data 704, or border 708 can result in movement of the end effector 408 in the depth direction at the second speed for fine movement. In some embodiments, a single-finger swipe in one or more of the map 702, imaging data 704, or border 708 can result in movement of the end effector 408 in the planar direction at a first speed for coarse movement. In some embodiments, a two-finger swipe in one or more of the map 702, imaging data 704, or border 708 can result in movement of the end effector 408 in the planar direction at a second speed for fine movement, where the first speed is faster than the second speed.

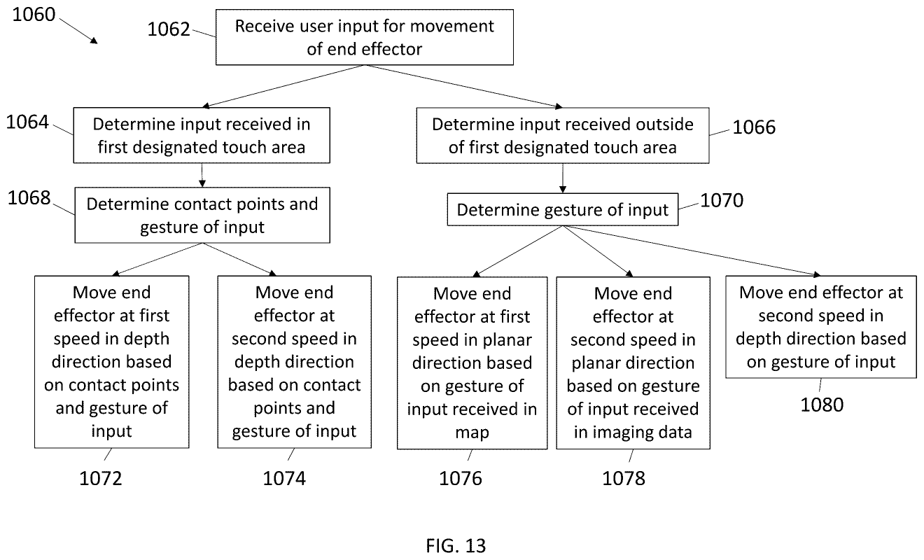

Referring now to FIG. 13, an example method 1060 of moving the end effector 408 at variable speeds is depicted. For instance, the driver functional facility can be configured to move the end effector with coarse control (e.g., a faster speed for larger movements) or with fine control (e.g., a slower speed for smaller, more precise movements), based on input received from a user by the input functional facility. In step 1062, the system 100 can receive input from the user (e.g., a surgeon) for moving the end effector 408. For instance, the input functional facility can receive input from the user for moving the end effector 408. In some embodiments, the input can be provided via a touch screen on the display 506. In some embodiments, the system 100, for instance the input functional facility, can determine where on the display 506 the input was received. In a step 1064 of the method 1060, the system 100, for instance the input functional facility, can determine that the input is received in the first designated touch area 1002. Input received in the first designated touch area 1002 can be for controlling movement of the end effector 408 in the depth direction. The depth direction can be toward or away from the tissue in front of the end effector 408. In some embodiments, the depth direction can be perpendicular to the tissue in front of the end effector 408. The depth direction can be toward or away from the tissue displayed in the imaging data 704. In some embodiments, the depth direction can be perpendicular to the tissue displayed in the imaging data 704. It should be appreciated, that in some embodiments, the tissue that is shown in the imaging data 704 is the tissue in front of the end effector 408. The depth direction can be along the longitudinal axis of the end effector 408. The depth direction can be the direction of the z-axis of the coordinate axes shown in FIG. 9 or FIG. 10. It should be understood that the z-axis of the coordinate axes shown in FIG. 9 and FIG. 10, shown as the block dot in the coordinate axes, is coming into and out of the page. In some embodiments, the input received in the first designated touch area 1002 can be the user swiping (e.g., dragging his finger) in the first designated touch area 1002. In some embodiments, swiping in a first direction can provide instruction to move the end effector 408 toward the tissue in front of the end effector 408 or in the imaging data 704, and swiping in a second direction (e.g., opposite the first direction) can provide instruction to move the end effector 408 away from the tissue in front of the end effector 408 or in the imaging data 704. In some embodiments, input received in the first designated touch area 1002 can have no effect on movement of the end effector in the planar direction.

In some embodiments, in step 1068, the system 100, and for instance the input functional facility, can determine the gesture and contact points of the input received in the first designated touch area 1002. Determining the gesture can include, in some embodiments, determining that the input received in the first designated touch area 1002 is a finger swipe. Determining the contact points can, in some embodiments, include determining the number of contact points of the input received in the first designated touch area 1002. For example, the contact points can be a single contact point when the user drags a single finger on the touch screen in the first designated touch area 1002. As another example, the contact points can be two contact points when the user drags two fingers on the touch screen in the first designated touch area 1002.

In some embodiments, the system 100 can move the end effector 408 in the depth direction when a finger swipe gesture is recognized as the input in the first designated touch area 1002. In some embodiments, the system 100 can move the end effector 408 at different speeds in the depth direction depending on the contact points of the input received from the user. In some embodiments, in step 1072, the system 100 can move the end effector 408 at a first speed in the depth direction based on the contact points of the input received in the first designated touch area 1002. In some embodiments, the first speed can be a speed for coarse movement of the end effector 408. In some embodiments, the system 100 can move the end effector 408 at the first speed in the depth direction when the input received in the first designated touch area 1002 includes a single contact point (e.g., a single-finger swipe). In some embodiments, in step 1074, the system 100 can move the end effector 408 at a second speed in the depth direction based on the contact points of the input received in the first designated touch area 1002. In some embodiments, the second speed can be a speed for fine movement of the end effector 408. In some embodiments, the first speed is greater than the second speed. In some embodiments, the system 100 can move the end effector 408 at the second speed in the depth direction when the input received in the first designated touch area 1002 includes two contact points (e.g., a two-finger swipe). In some embodiments, the input functional facility can provide instruction to move the end effector 408 in the depth direction based on the gesture and contact points of the input received in the first designated touch area 1002. In some embodiments, the driver functional facility can move the end effector 408 in the depth direction based on the gesture and contact points of the input received in the first designated touch area 1002.

In a step 1066 of the method 1030, the system 100, for instance the input functional facility, can determine that the input is received outside the first designated touch area 1002. Input received outside the first designated touch area 1002 can be for controlling movement of the end effector 408 in the planar direction or in the depth direction. The planar direction can generally be in the plane perpendicular to the depth direction. The planar direction can be in the plane parallel to the tissue in front of the end effector 408. The planar direction can in the plane parallel to the tissue displayed in the imaging data 704. It should be appreciated, that in some embodiments, the tissue that is shown in the imaging data 704 is the tissue in front of the end effector 408. The planar direction can be in the plane parallel to the longitudinal axis of the end effector 408. The planar direction can be the x-y plane of the coordinate axes shown in FIG. 9 or FIG. 10. Movement in the planar direction can involve moving the end effector 408 up, down, left, or right in the surgical site without moving the end effector 408 toward or away from the tissue in front of the end effector 408, for instance.

In some embodiments, the input received outside the first designated touch area 1002 can be the user swiping (e.g., dragging his finger) in the imaging data 704. In some embodiments, the input received outside the first designated touch area 1002 can be the user swiping (e.g., dragging his finger) in the map 702. In some embodiments, the input received outside the first designated touch area 1002 can be the user swiping (e.g., dragging his finger) in the border 708. In some embodiments, the input received outside the first designated touch area 1002 can be the user swiping (e.g., dragging his finger) in the imaging data 704 and the border 708. For instance, in some embodiments, the user can begin a swiping motion in the imaging data 704 and continue the swiping motion into the border 708. In some embodiments, the input received outside the first designated touch area 1002 can be the user pinching (e.g., dragging two fingers toward one another, or dragging two fingers away from one another) in the imaging data 704. In some embodiments, the input received outside the first designated touch area 1002 can be the user pinching (e.g., dragging two fingers toward or away from one another) in the map 702. In some embodiments, the input received outside the first designated touch area 1002 can be the user pinching (e.g., dragging two fingers toward or away from one another) in the border 708. In some embodiments, the input received outside the first designated touch area 1002 can be the user pinching (e.g., dragging two fingers toward or away from one another) in two or more of the imaging data 704, the map 702, or the border 708. For instance, in some embodiments, the user can begin a pinching motion in the map 702 and continue the pinching motion through the border 708 and into the imaging data 704.

In some embodiments, for the system 100, particularly for instance the input functional facility, to recognize an instruction for planar or depth movement of the end effector 408 outside of the first designated touch area 1002, the input must be input by the user in the second designated touch area 1004 (e.g., pinching his fingers in the second designated touch area 1004, swiping his fingers in a region of the map 702 within the second designated touch area 1004, or swiping his fingers in a region of the imaging data 704 or border 708 within the second designated touch area 1004). In some embodiments, providing an input on the user interface outside the first designated touch area 1002 and the second designated touch area 1004 is not registered as an instruction for movement of the end effector 408. In some embodiments, depending on the direction of swiping in the x-y plane of the coordinate axes of FIG. 9 and FIG. 10, an instruction can be provided for moving the end effector up/down (e.g., in the direction of the y-axis of the coordinate axes of FIG. 9 and FIG. 10), left/right (e.g., in the direction of the x-axis of the coordinate axes of FIG. 9 and FIG. 10), or both up/down and left/right. In some embodiments, a swiping input received outside the first designated touch area 1002 can have no effect on movement of the end effector 408 in the depth direction. In some embodiments, depending on the direction of pinching gesture of the input (e.g., moving two fingers toward each other or away from each other), an instruction can be provided for moving the end effector 408 in the depth direction toward or away from the tissue in front of the end effector 408 or in the imaging data 704. For instance, a pinching gesture in which two fingers are dragged away from each other can result in moving the end effector toward the tissue in front of the end effector 408 or in the imaging data 704, and a pinching gesture in which two fingers are dragged toward each other can result in moving the end effector away from the tissue in front of the end effector 408 or in the imaging data 704. In some embodiments, a pinching input received outside the first designated touch area 1002 can have no effect on movement of the end effector in the planar direction.

In some embodiments, in step 1070, the system 100, and for instance the input functional facility, can determine the gesture of the input received outside the first designated touch area 1002. Determining the gesture can include, in some embodiments, determining whether the input received outside the first designated touch area 1002 is a finger swipe or a pinch. For instance, a swipe can be recognized by a single contact point moving across the display (e.g., a single-finger swipe) or multiple contact points moving across the screen substantially in the same directions (e.g., a two-finger swipe). A pinch can be recognized by two contact points moving roughly parallel and in opposite directions to one another (e.g., two fingers being brought to together or moved away from each other).

In some embodiments, the system 100 can move the end effector 408 in the planar direction when a finger swipe gesture is recognized as the input outside the first designated area 1002. In some embodiments, the system 100 can move the end effector 408 at different speeds in the planar direction depending on where the input is received from the user outside the first designated touch area 1002. In some embodiments, in step 1076, the system 100 can move the end effector 408 at a first speed in the planar direction based on the input received being in the map 702. In some embodiments, the first speed can be a speed for coarse movement of the end effector 408. In some embodiments, the system 100 can move the end effector 408 at the first speed in the planar direction when the input received in the map 702 includes a finger swipe (e.g., one-finger swipe, two-finger swipe, or any number of fingers swipe). In some embodiments, in step 1078, the system 100 can move the end effector 408 at a second speed in the planar direction based on the input being received being in the imaging data 704 or the border 708. In some embodiments, the second speed can be a speed for fine movement of the end effector 408. In some embodiments, the first speed is faster than the second speed. In some embodiments, the system 100 can move the end effector 408 at the second speed in the planar direction when the input received in the imaging data 704 or the border 708 includes a finger swipe (e.g., one-finger swipe, two-finger swipe, or any number of fingers swipe). In some embodiments, the input functional facility can provide instruction to move the end effector 408 in the planar direction based on the gesture and location of the input received outside the first designated touch area 1002. In some embodiments, the driver functional facility can move the end effector 408 in the planar direction based on the gesture and location of the input received outside the first designated touch area 1002.

In some embodiments, the system 100 can move the end effector 408 in the depth direction when a pinch gesture is recognized as the input outside the first designated area 1002. In some embodiments, in step 1080, the system 100 can move the end effector 408 at the second speed in the depth direction based on the gesture of the input received outside the first designated touch area 1002 (e.g., a pinching gesture received outside the first designated touch area 1002). In some embodiments, the input functional facility can provide instruction to move the end effector 408 in the depth direction based on the gesture of the input received outside the first designated touch area 1002. In some embodiments, the driver functional facility can move the end effector 408 in the depth direction based on the gesture of the input received outside the first designated touch area 1002.

In some embodiments, a single-finger swipe in the first designated touch area 1002 can result in movement of the end effector 408 in the depth direction at a first speed for coarse movement. In some embodiments, a two-finger swipe in the first designated touch area 1002 can result in movement of the end effector 408 in the depth direction at a second speed for fine movement, where the first speed is faster than the second speed. In some embodiments, a pinch gesture in one or more of the map 702, imaging data 704, or border 708 can result in movement of the end effector 408 in the depth direction at the second speed for fine movement. In some embodiments, a finger swipe (e.g., any number of fingers) in the map 702 can result in movement of the end effector 408 in the planar direction at a first speed for coarse movement. In some embodiments, a finger swipe (e.g., any number of fingers) in one or more of the imaging data 704 or border 708 can result in movement of the end effector 408 in the planar direction at a second speed for fine movement, where the first speed is faster than the second speed.

In some embodiments, user input received in the user interface 506 can control the rotation of the end effector 408. That is, the input functional facility can recognize the input provided by the user and instruct the drive functional facility to rotate the end effector 408 accordingly. Rotating the end effector 408 can include rotating the end effector 408 about the axis defining the depth direction (e.g., the z-axis in the coordinate axes of FIGS. 9 and 10, which is shown as the black dot and should be understood as coming into and out of the page). The axis defining the depth direction can be perpendicular to the plane defining the planar direction (e.g., the x-y plane of the coordinate axes of FIGS. 9 and 10). In some embodiments, the axis defining the depth direction can be the longitudinal axis of the end effector 408. The user can input to rotate the end effector 408 can be a twisting gesture provided on the user interface. The twisting gesture can involve creating two or more contact points on the user interface (e.g., placing two or more fingers against the touch screen of the user interface), and rotating the contact points about one another (e.g., the user rotating his hand while two or more fingers are placed against the touch screen). Depending on the location of two or more contact points on the user interface, the end effector 408 can be rotated about the axis defining the depth direction at different speeds (e.g., a first, faster speed for coarse rotation, and a second, slower speed for fine rotation). In some embodiments, if the contact points for the twisting gesture are in the first designated touch area 1002, the rotation of the end effector can be at a second speed for fine rotation. In some embodiments, if the contact points for the twisting gesture are outside the first designated touch area 1002 (e.g., in one or more of the map 702, imaging data 704, or border 708), the rotation of the end effector can be at a first speed for coarse rotation. In some embodiments, if the contact points for the twisting gesture are in the second designated touch area 1004, the rotation of the end effector can be at a first speed for coarse rotation. In some embodiments, if the contact points for the twisting gesture are in one or more of the imaging data 704 or border 708, the rotation of the end effector can be at a second speed for fine rotation, and if the contact points for the twisting gesture are in the map 702, the rotation of the end effector can be at a first speed for coarse rotation.

In some embodiments, the various steps of the method 1010, the method 1030, or the method 1060 can completed by the console cart 102 or shared between the console cart 102 and the robotic cart 104. For instance, it should be appreciated that the console cart 102 can generate an instruction to move the end effector 408 and the robotic cart 104 can execute the instruction. Moreover it should be appreciated while the above-described methods were discussed with respect to the inputs from the user being provided as finger gestures (e.g., finger swipes or pinches) on the user interface, this is a non-limiting example. For instance, the methods can be performed in the same manner when the inputs from the user are provided as stylus gestures (moving one or more styluses) on the user interface. In the methods 1010, 1030, and 1060 discussed above, the user end effector 408 can be freely moved in both the planar and depth directions based on input received from the user.

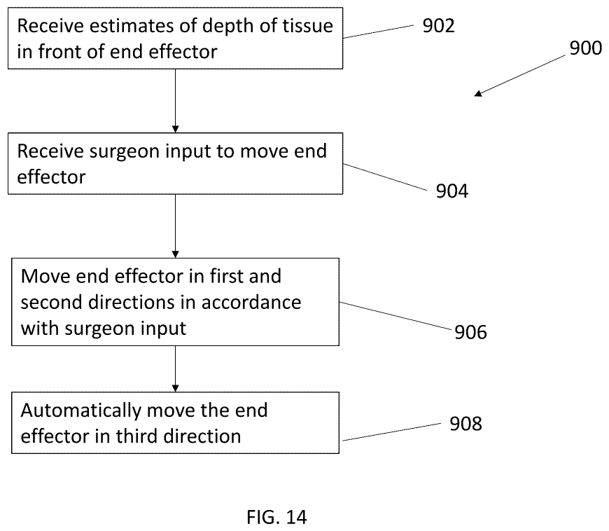

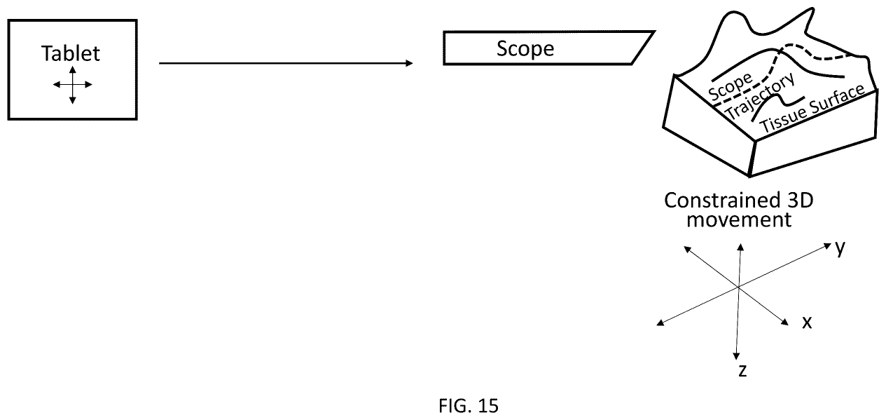

Referring now to FIG. 14, a method 900 of semi-autonomously moving the end effector 408 is depicted. In step 902, the system 100 can receive estimates of the depth of tissue in front of the end effector 408 (e.g., the distance between the end effector 408 and the tissue in front of the end effector 408). The “depth” measurement can be in the Z direction of the coordinate axes shown in FIGS. 7, 9, 10, and 15. It should be understood that the z-axis of the coordinate axes shown in FIG. 7, FIG. 9, and FIG. 10, shown as the black dot in the coordinate axes, is coming into and out of the page. In step 904, the system 100 can receive user input to move the end effector 408 in the planar direction, as discussed with respect to the methods 800, 1010, 1030, and 1060. In step 906, the system 100 can move the end effector 408 freely in the planar direction (e.g., in first and second directions) in accordance with the user input. The first and second directions can be the X an Y directions of the coordinate axes shown in FIGS. 7, 9, 10, and 15. The first and second directions can be in the plane defining the planar direction. The third direction can be the depth direction. In some embodiments, the third direction can be parallel to the plane of tissue in front of the end effector 408. The first and second directions can lie in the two-dimensional plane that the real-time imaging data 704 is depicted in on the display 506. Accordingly, the surgeon can easily provide input to move the end effector 408 in the first and second directions (e.g., by moving his finger along the surface of the display 506). In some embodiments, in step 908 of the method 900, the system 100 can automatically move the end effector 408 in the third direction (e.g., the depth direction). The end effector 408 can automatically be moved in the third direction to maintain a constant distance between the end effector 408 and the tissue in the third direction (e.g., the tissue in front of the end effector 408). The system 100 can move the end effector 408 in relation the contours of the tissue in the third direction to maintain the constant distance, as shown in FIG. 15. The system 100 can therefore maintain a “virtual bumper” between the end effector 408 and the tissue in front of the end effector 408. The virtual bumper can be in place at all times during operation of the system 100. In some embodiments, the virtual bumper can be implemented only when the end effector 408 is being actuated (e.g., enucleating, aspirating, cutting, etc.). In some embodiments, the virtual bumper can be set by a user (e.g., the sets the distance to be maintained between the end effector 408 and tissue in front of the end effector 408). In some embodiments, the length of the virtual bumper can vary based on whether the end effector 408 is being moved while being actuated or not actuated. In some embodiments, the length of the virtual bumper can vary based on the position of the end effector 408 in the surgical site. It should be appreciated that in some embodiments, the various steps of the method 900 can completed between the console cart 102 and the robotic cart 104. For instance, the console cart 102 can receive surgeon input and generate instructions for the robotic cart 104. which can then control the movement of the end effector 408.

In some embodiments, the surgical site discussed above can be the prostate.

Techniques operating according to the principles described herein may be implemented in any suitable manner. Included in the discussion above are flow charts showing the steps and acts of various processes that move an end effector in one or more directions and at one or more speeds. The processing and decision blocks of the flow charts above represent steps and acts that may be included in algorithms that carry out these various processes. Algorithms derived from these processes may be implemented as software integrated with and directing the operation of one or more single-or multi-purpose processors, may be implemented as functionally-equivalent circuits such as a Digital Signal Processing (DSP) circuit. Field Programmable Gate Array (FPGA), or an Application-Specific Integrated Circuit (ASIC), or may be implemented in any other suitable manner. It should be appreciated that the flow charts included herein do not depict the syntax or operation of any particular circuit or of any particular programming language or type of programming language. Rather, the flow charts illustrate the functional information one of ordinary skill in the art may use to fabricate circuits or to implement computer software algorithms to perform the processing of a particular apparatus carrying out the types of techniques described herein. It should also be appreciated that, unless otherwise indicated herein, the particular sequence of steps and/or acts described in each flow chart is merely illustrative of the algorithms that may be implemented and can be varied in implementations and embodiments of the principles described herein.

Accordingly, in some embodiments, the techniques described herein may be embodied in computer-executable instructions implemented as software, including as application software, system software, firmware, middleware, embedded code, or any other suitable type of software. Such computer-executable instructions may be written using any of a number of suitable programming languages and/or programming or scripting tools, and also may be compiled as executable machine language code or intermediate code that is executed on a framework or virtual machine.

When techniques described herein are embodied as computer-executable instructions, these computer-executable instructions may be implemented in any suitable manner, including as a number of functional facilities, each providing one or more operations to complete execution of algorithms operating according to these techniques. A “functional facility.” however instantiated, is a structural component of a computer system that, when integrated with and executed by one or more computers, causes the one or more computers to perform a specific operational role. A functional facility may be a portion of or an entire software element. For example, a functional facility may be implemented as a function of a process, or as a discrete process, or as any other suitable unit of processing. If techniques described herein are implemented as multiple functional facilities, each functional facility may be implemented in its own way; all need not be implemented the same way. Additionally, these functional facilities may be executed in parallel and/or serially, as appropriate, and may pass information between one another using a shared memory on the computer(s) on which they are executing, using a message passing protocol, or in any other suitable way.

Generally, functional facilities include routines, programs, objects, components, data structures, etc. that perform particular tasks or implement particular abstract data types. Typically, the functionality of the functional facilities may be combined or distributed as desired in the systems in which they operate. In some implementations, one or more functional facilities carrying out techniques herein may together form a complete software package. These functional facilities may, in alternative embodiments, be adapted to interact with other, unrelated functional facilities and/or processes, to implement a software program application.

Some exemplary functional facilities have been described herein for carrying out one or more tasks. It should be appreciated, though, that the functional facilities and division of tasks described is merely illustrative of the type of functional facilities that may implement the exemplary techniques described herein, and that embodiments are not limited to being implemented in any specific number, division, or type of functional facilities. In some implementations, all functionality may be implemented in a single functional facility. It should also be appreciated that, in some implementations, some of the functional facilities described herein may be implemented together with or separately from others (i.e., as a single unit or separate units), or some of these functional facilities may not be implemented.

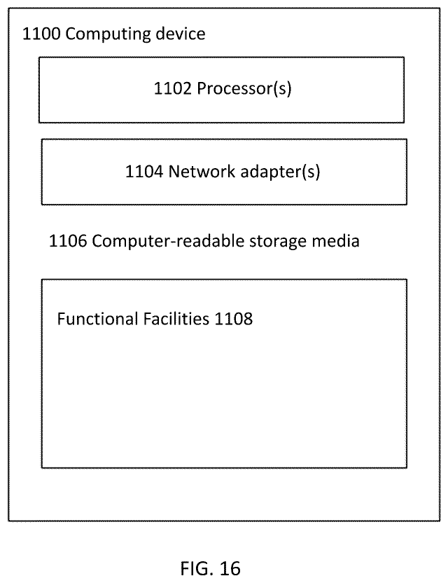

Computer-executable instructions implementing the techniques described herein (when implemented as one or more functional facilities or in any other manner) may, in some embodiments, be encoded on one or more computer-readable media to provide functionality to the media. Computer-readable media include magnetic media such as a hard disk drive, optical media such as a Compact Disk (CD) or a Digital Versatile Disk (DVD), a persistent or non-persistent solid-state memory (e.g., Flash memory, Magnetic RAM, etc.), or any other suitable storage media. Such a computer-readable medium may be implemented in any suitable manner, including as computer-readable storage media 1106 of FIG. 16 described below (i.e., as a portion of a computing device 1100) or as a stand-alone, separate storage medium. As used herein, “computer-readable media” (also called “computer-readable storage media”) refers to tangible storage media. Tangible storage media are non-transitory and have at least one physical, structural component. In a “computer-readable medium.” as used herein, at least one physical, structural component has at least one physical property that may be altered in some way during a process of creating the medium with embedded information, a process of recording information thereon, or any other process of encoding the medium with information. For example, a magnetization state of a portion of a physical structure of a computer-readable medium may be altered during a recording process.

In some, but not all, implementations in which the techniques may be embodied as computer-executable instructions, these instructions may be executed on one or more suitable computing device(s) operating in any suitable computer system, including the exemplary computing system of FIG. 3 or 5, or one or more computing devices (or one or more processors of one or more computing devices) may be programmed to execute the computer-executable instructions. A computing device or processor may be programmed to execute instructions when the instructions are stored in a manner accessible to the computing device/processor, such as in a local memory (e.g., an on-chip cache or instruction register, a computer-readable storage medium accessible via a bus, a computer-readable storage medium accessible via one or more networks and accessible by the device/processor, etc.). Functional facilities that comprise these computer-executable instructions may be integrated with and direct the operation of a single multi-purpose programmable digital computer apparatus, a coordinated system of two or more multi-purpose computer apparatuses sharing processing power and jointly carrying out the techniques described herein, a single computer apparatus or coordinated system of computer apparatuses (co-located or geographically distributed) dedicated to executing the techniques described herein, one or more Field-Programmable Gate Arrays (FPGAs) for carrying out the techniques described herein, or any other suitable system.

FIG. 16 is a block diagram of an example computing system for receiving user input for moving an end effector and controlling a robotic arm to move the end effector in a certain direction and speed, in accordance with the input, that can be used in performing surgical techniques. FIG. 16 illustrates one possible implementation of a computing device in the form of a computing device 1100 that may be used in a system implementing techniques described herein, although others are possible. It should be appreciated that FIG. 16 is intended neither to be a depiction of necessary components for a computing device to adjust a position of an imaginary pivot point along an end effector, nor a comprehensive depiction.

Computing device 1100 may include at least one processor 1102 (e.g., a computer hardware processor), a network adapter 1104, and computer-readable storage media 1106. Computing device 1100 may be, for example, a desktop or laptop personal computer, a personal digital assistant (PDA), a smart mobile phone, or any other suitable computing device. Network adapter 1104 may be any suitable hardware and/or software to enable the computing device 1100 to communicate wired and/or wirelessly with any other suitable computing device over any suitable computing network. The computing network may include wireless access points, switches, routers, gateways, and/or other networking equipment as well as any suitable wired and/or wireless communication medium or media for exchanging data between two or more computers, including the Internet. Computer-readable storage media 1106 may be adapted to store data to be processed and/or instructions to be executed by processor 1102. Processor 1102 enables processing of data and execution of instructions. The data and instructions may be stored on the computer-readable storage media 1106.

The data and instructions stored on computer-readable storage media 1106 may comprise computer-executable instructions implementing techniques which operate according to the principles described herein. In the example of FIG. 16, computer-readable storage media 1106 stores computer-executable instructions implementing various facilities 1108 and storing various information as described above. Computer-readable storage media 1106 may store a driver facility configured to perform techniques described herein, such as actuating a robot arm to move an end effector. Computer-readable storage media 1106 may store an actuation facility configured to perform techniques described herein, such as actuating an end effector. Computer-readable storage media 1106 may store an imaging facility configured to perform techniques described herein, such as gathering imaging data of a surgical site. Computer-readable storage media 1106 may store a locating facility configured to perform techniques described herein, such as determining a location of an end effector. Computer-readable storage media 1106 may store a landmark facility configured to perform techniques described herein, such as identifying one or more anatomical landmarks in the surgical site or imaging data. Computer-readable storage media 1106 may store a relative locating facility configured to perform techniques described herein, such as determining a location of the end effector relative tissue in the surgical site. Computer-readable storage media 1106 may store a mapping facility configured to perform techniques described herein, such generating a map of the surgical site. Computer-readable storage media 1106 may store a display facility configured to perform techniques described herein, such as generating a display on a user interface including imaging data or a map of the surgical site. Computer-readable storage media 1106 may store an input facility configured to perform techniques described herein, such as receiving input from a user. for instance on the user interface, and generating an instruction in accordance with the input. Computer-readable storage media 1106 may store a depth movement facility configured to perform techniques described herein, such as automatically controlling movement of the end effector in the depth direction.

While not illustrated in FIG. 16, a computing device may additionally have one or more components and peripherals, including input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets.

Embodiments have been described where the techniques are implemented in circuitry and/or computer-executable instructions. It should be appreciated that some embodiments may be in the form of a method, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

In some aspects, the techniques described herein relate to a system, including: a surgical robot, including a robot arm configured to hold an end effector; and one or more processing devices configured to: receive an estimate of a position or a location of the end effector in a surgical site; receive an estimate of a depth of tissue in front of the end effector; generate a map of the surgical site, wherein the map extends beyond a field of view of real-time imaging data of the surgical site generated at a current position or location of the end effector; and present the map of the surgical site and the real-time imaging data on a display.

In some aspects, the techniques described herein relate to a system, wherein the map of the surgical site shows a contour of a tissue of the surgical site.

In some aspects, the techniques described herein relate to a system, wherein the real-time imaging data is positioned on the map at the current position or location of the end effector.

In some aspects, the techniques described herein relate to a system, wherein the real-time imaging data is displayed in a border that indicates a direction and a magnitude of a force acting on the end effector.

In some aspects, the techniques described herein relate to a system, wherein the one or more processing devices are further configured to: receive input on the display for moving the end effector; determine if the input is received in the map of the surgical site or in an end effector marker shown on the display; move the end effector at a first speed if the input is received in the map of the surgical site; and move the end effector at a second speed if the input is received in the end effector marker.

In some aspects, the techniques described herein relate to a system, wherein the end effector marker is a fixed distance from the real-time imaging data on the display.

In some aspects, the techniques described herein relate to a system, including: a surgical robot, including a robot arm configured to hold an end effector; and one or more processing devices configured to: receive an estimate of a depth of tissue in front of the end effector; receive input to move the end effector in first and second directions; generate an instruction to move the end effector in first and second directions in accordance with the received input; and automatically generate an instruction to move the end effector in a third direction.

In some aspects, the techniques described herein relate to a system, wherein: the first and second directions are parallel to a plane of the tissue in front of the end effector; and the third direction is perpendicular to the plane of the tissue in front of the end effector.

In some aspects, the techniques described herein relate to a system, wherein the instruction to move the end effector in the third direction is to maintain a set distance between the end effector and the tissue in the third direction

Non-limiting embodiments of the present disclosure are set out in the following clauses:

Clause 1. A system, comprising: a surgical robot, comprising a robot arm configured to hold an end effector; and at least one circuit configured to: generate a presentation to be presented on a user interface, wherein the presentation comprises: imaging data of a surgical site; a map of an anatomy of the surgical site surrounding the imaging data; and a first designated touch area; receive a user input on the user interface; and actuate the robot arm to move the end effector in one of a depth direction or a planar direction based on the user input.

Clause 2. The system of clause 1, wherein the surgical site comprises a prostate.

Clause 3. The system of clause 1 or clause 2, wherein the at least one circuit is configured to actuate the robot arm to move the end effector in one of a depth direction or a planar direction based on a location of the received user input on the user interface.

Clause 4. The system of any one of clauses 1-3, wherein, when the received user input is in the first designated touch area, the at least one circuit is configured to actuate the robot arm to move the end effector in the depth direction based on the user input received in the first designated touch area.

Clause 5. The system of any one of clauses 1-4, wherein the at least one circuit is configured to actuate the robot arm to move the end effector in the depth direction with a speed based on a speed of the user input received in the first designated touch area.

Clause 6. The system of any one of clauses 1-5, wherein: the received user input comprises one or more contact points with the user interface; and the at least one circuit is configured to actuate the robot arm to move the end effector in the depth direction with a speed based on the one or more contact points.

Clause 7. The system of any one of clauses 1-6, wherein the at least one circuit is configured to: actuate the robot arm to move the end effector in the depth direction with a first speed when the user input comprises one contact point; and actuate the robot arm to move the end effector in the depth direction with a second speed when the user input comprises a plurality of contact points.

Clause 8. The system of any one of clauses 1-7, wherein, when the received user input is outside the first designated touch area, the at least one circuit is configured to actuate the robot arm to move the end effector in the planar direction based on the user input received outside the first designated touch area.

Clause 9. The system of any one of clauses 1-8, wherein the at least one circuit is configured to actuate the robot arm to move the end effector in the planar direction with a speed based on a speed of the user input received outside the first designated touch area.

Clause 10. The system of any one of clauses 1-9, wherein: the received user input comprises one or more contact points with the user interface; and the at least one circuit is configured to actuate the robot arm to move the end effector in the planar direction with a speed based on the one or more contact points.

Clause 11. The system of any one of clauses 1-10, wherein the at least one circuit is configured to: actuate the robot arm to move the end effector in the planar direction with a first speed when the user input comprises one contact point; and actuate the robot arm to move the end effector in the planar direction with a second speed when the user input comprises a plurality of contact points.

Clause 12. The system of any one of clauses 1-11, wherein the received user input is in a second designated touch area, wherein the second designated touch area comprises at least a portion of the imaging data and at least a portion of the map.

Clause 13. The system of any one of clauses 1-12, wherein, when the received user input is in at least one of the imaging data or the map, the at least one circuit is configured to actuate the robot arm to move the end effector in the planar direction based on the user input received in the at least one of the imaging data or the map.

Clause 14. The system of any one of clauses 1-13, wherein the at least one circuit is configured to actuate the robot arm to move the end effector in the planar direction with a speed based on a speed of the user input received in at least one of the imaging data or the map.

Clause 15. The system of any one of clauses 1-14, wherein: the received user input comprises one or more contact points with the user interface; and the at least one circuit is configured to actuate the robot arm to move the end effector in the planar direction with a speed based on the one or more contact points.

Clause 16. The system of any one of clauses 1-15, wherein the at least one circuit is configured to: actuate the robot arm to move the end effector in the planar direction with a first speed when the user input comprises one contact point; and actuate the robot arm to move the end effector in the planar direction with a second speed when the user input comprises a plurality of contact points.

Clause 17. The system of any one of clauses 1-16, wherein the at least one circuit is configured to: actuate the robot arm to move the end effector in the planar direction with a first speed when the user input is received in the map; and actuate the robot arm to move the end effector in the planar direction with a second speed when the user input is received in the imaging data.

Clause 18. The system of any one of clauses 1-17, wherein the planar direction is defined in a plane, and the depth direction is perpendicular to the plane.

Clause 19. The system of any one of clauses 1-18, wherein the user input is a swiping gesture on the user interface.

Clause 20. The system of any one of clauses 1-19, wherein the at least one circuit is configured to actuate the robot arm to move the end effector in one of a depth direction or a planar direction based on a location of the user input received on the user interface and a gesture of the user input received on the user interface.

Clause 21. The system of any one of clauses 1-20, wherein the at least one circuit is configured to: actuate the robot arm to move the end effector in the depth direction when the received user input is a first gesture received in the first designated touch area; actuate the robot arm to move the end effector in the planar direction when the received user input is the first gesture received outside the first designated touch area; and actuate the robot arm to move the end effector in the depth direction when the received user input is a second gesture received outside the first designated touch area.

Clause 22. The system of any one of clauses 1-21, wherein the at least one circuit is configured to actuate the robot arm to move the end effector in the depth direction at a speed for fine movement when the received user input is a second gesture received outside the first designated touch area.

Clause 23. The system of any one of clauses 1-22, wherein the planar direction is defined in a plane, and the depth direction is perpendicular to the plane.

Clause 24. The system of any one of clauses 1-23, wherein the first gesture is a swiping gesture on the user interface, and wherein the second gesture is a pinching gesture on the user interface.

Clause 25. A system, comprising: a surgical robot, comprising a robot arm configured to hold an end effector; and at least one circuit configured to: generate a presentation to be presented on a user interface, wherein the presentation comprises: imaging data of a surgical site; and a map of an anatomy of the surgical site surrounding the imaging data; receive a user input on the user interface; and actuate the robot arm to move the end effector with a speed based on the user input.

Clause 26. The system of clause 25, wherein the at least one circuit is configured to actuate the robot arm to move the end effector with a speed based on a speed of the user input received.

Clause 27. The system of clause 25 or clause 26, wherein: the received user input comprises one or more contact points with the user interface; and the at least one circuit is configured to actuate the robot arm to move the end effector with a speed based on the one or more contact points.

Clause 28. The system of any one of clauses 25-27, wherein the at least one circuit is configured to: actuate the robot arm to move the end effector with a first speed when the user input comprises one contact point; and actuate the robot arm to move the end effector with a second speed when the user input comprises a plurality of contact points.

Clause 29. The system of any one of clauses 25-28, wherein the at least one circuit is configured to actuate the robot arm to move the end effector with a speed based on a location of the received user input on the user interface.

Clause 30. The system of any one of clauses 25-29, wherein the at least one circuit is configured to: actuate the robot arm to move the end effector with a first speed when the user input is received in the map; and actuate the robot arm to move the end effector with a second speed when the user input is received in the imaging data.

Clause 31. The system of any one of clauses 25-30, wherein the at least one circuit is configured to actuate the robot arm to move the end effector with a speed based on a gesture of the user input received.

Clause 32. The system of any one of clauses 25-31, wherein the at least one circuit is configured to: actuate the robot arm to move the end effector at a first speed when the received user input is a first gesture; and actuate the robot arm to move the end effector at a second speed when the received user input is a second gesture.

Clause 33. The system of any one of clauses 25-32, wherein the first gesture is a swiping gesture, and the second gesture is a pinching gesture.

Clause 34. A method, comprising: receiving imaging data of a surgical site, wherein a robot arm is configured to position an end effector in the surgical site; generating a presentation to be presented on a user interface, wherein the presentation comprises: imaging data of the surgical site; a map of an anatomy of the surgical site surrounding the imaging data; and a first designated touch area; receiving a user input on a user interface; and actuating the robot arm to move the end effector in one of a depth direction or a planar direction based on the user input.

Clause 35. The method of clause 34, further comprising actuating the robot arm to move the end effector in one of a depth direction or a planar direction based on a location of the user input received on the user interface.

Clause 36. The method of clause 34 or clause 35, further comprising, when the received user input is in the first designated touch area, moving the end effector in the depth direction based on the user input received in the first designated touch area.

Clause 37. The method of any one of clauses 34-36, further comprising actuating the robot arm to move the end effector in the depth direction with a speed based on a speed of the user input received in the first designated touch area.

Clause 38. The method of any one of clauses 34-37, further comprising actuating the robot arm to move the end effector in the depth direction with a speed based on one or more contact points of the received user input with the user interface.

Clause 39. The method of any one of clauses 34-38, further comprising: actuating the robot arm to move the end effector in the depth direction with a first speed when the user input comprises one contact point; and actuating the robot arm to move the end effector in the depth direction with a second speed when the user input comprises a plurality of contact points.

Clause 40. The method of any one of clauses 34-39, further comprising, when the received user input is outside the first designated touch area, actuating the robot arm to move the end effector in the planar direction based on the user input received outside the first designated touch area.

Clause 41. The method of any one of clauses 34-40, further comprising actuating the robot arm to move the end effector in the planar direction with a speed based on a speed of the user input received outside the first designated touch area.

Clause 42. The method of any one of clauses 34-41, further comprising actuating the robot arm to move the end effector in the planar direction with a speed based on one or more contact points of the received user input with the user interface.

Clause 43. The method of any one of clauses 34-42, further comprising: actuating the robot arm to move the end effector in the planar direction with a first speed when the user input comprises one contact point; and actuating the robot arm to move the end effector in the planar direction with a second speed when the user input comprises a plurality of contact points.

Clause 44. The method of any one of clauses 34-43, wherein the received user input is in a second designated touch area, wherein the second designated touch area comprises at least a portion of the imaging data and at least a portion of the map.

Clause 45. The method of any one of clauses 34-44, further comprising, when the received user input is in at least one of the imaging data or the map, actuating the robot arm to move the end effector in the planar direction based on the user input received in the at least one of the imaging data or the map.

Clause 46. The method of any one of clauses 34-45, further comprising actuating the robot arm to move the end effector in the planar direction with a speed based on a speed of the user input received in at least one of the imaging data or the map.

Clause 47. The method of any one of clauses 34-46, further comprising actuating the robot arm to move the end effector in the planar direction with a speed based on one or more contact points of the received user input with the user interface.

Clause 48. The method of any one of clauses 34-47, further comprising: actuating the robot arm to move the end effector in the planar direction with a first speed when the user input comprises one contact point; and actuating the robot arm to move the end effector in the planar direction with a second speed when the user input comprises a plurality of contact points.

Clause 49. The method of any one of clauses 34-48, further comprising: actuating the robot arm to move the end effector in the planar direction with a first speed when the user input is received in the map; and actuating the robot arm to move the end effector in the planar direction with a second speed when the user input is received in the imaging data.

Clause 50. The method of any one of clauses 34-49, wherein the planar direction is defined in a plane, and the depth direction is perpendicular to the plane.

Clause 51. The method of any one of clauses 34-50, wherein the user input is a swiping gesture on the user interface.

Clause 52. The method of any one of clauses 34-51, further comprising actuating the robot arm to move the end effector in one of a depth direction or a planar direction based on a location of the user input received on the user interface and a gesture of the user input received on the user interface.

Clause 53. The method of any one of clauses 34-52, further comprising: actuating the robot arm to move the end effector in the depth direction when the received user input is a first gesture received in the first designated touch area; actuating the robot arm to move the end effector in the planar direction when the received user input is the first gesture received outside the first designated touch area; and actuating the robot arm to move the end effector in the depth direction when the received user input is a second gesture received outside the first designated touch area.

Clause 54. The method of any one of clauses 34-53, further comprising actuating the robot arm to move the end effector in the depth direction at a speed for fine movement when the received user input is a second gesture received outside the first designated touch area.

Clause 55. The method of any one of clauses 34-54, wherein the planar direction is defined in a plane, and the depth direction is perpendicular to the plane.

Clause 56. The method of any one of clauses 34-55, wherein the first gesture is a swiping gesture on the user interface, and wherein the second gesture is a pinching gesture on the user interface.

Clause 57. A method, comprising: receiving imaging data of a surgical site, wherein a robot arm is configured to position an end effector in the surgical site; generating a presentation to be presented on a user interface, wherein the presentation comprises: imaging data of the surgical site; and a map of an anatomy of the surgical site surrounding the imaging data; receiving a user input on the user interface; and actuating the robot arm to move the end effector with a speed based on the user input.

Clause 58. The method of clause 57, further comprising actuating the robot arm to move the end effector with a speed based on a speed of the user input received.

Clause 59. The method of clause 57 or clause 58, further comprising actuating the robot arm to move the end effector with a speed based on one or more contact points of the received user input on the user interface.

Clause 60. The method of any one of clauses 57-59, further comprising: actuating the robot arm to move the end effector with a first speed when the user input comprises one contact point; and actuating the robot arm to move the end effector with a second speed when the user input comprises a plurality of contact points.

Clause 61. The method of any one of clauses 57-60, further comprising actuating the robot arm to move the end effector with a speed based on a location of the received user input on the user interface.

Clause 62. The method of any one of clauses 57-61, further comprising: actuating the robot arm to move the end effector with a first speed when the user input is received in the map; and actuating the robot arm to move the end effector with a second speed when the user input is received in the imaging data.

Clause 63. The method of any one of clauses 57-62, further comprising actuating the robot arm to move the end effector with a speed based on a gesture of the user input received.

Clause 64. The method of any one of clauses 57-63, further comprising: actuating the robot arm to move the end effector at a first speed when the received user input is a first gesture; and actuating the robot arm to move the end effector at a second speed when the received user input is a second gesture.

Clause 65. The method of any one of clauses 57-64, wherein the first gesture is a swiping gesture, and the second gesture is a pinching gesture.

Clause 66. At least one non-transitory computer-readable storage medium having encoded thereon executable instructions that, when executed by at least one processor, cause the at least one processor to: receive imaging data of a surgical site, wherein a robot arm is configured to position an end effector in the surgical site; generate a presentation to be presented on a user interface, wherein the presentation comprises: imaging data of the surgical site; a map of an anatomy of the surgical site surrounding the imaging data; and a first designated touch area; receive a user input on a user interface; and actuate the robot arm to move the end effector in one of a depth direction or a planar direction based on the user input.

Clause 67. At least one non-transitory computer-readable storage medium having encoded thereon executable instructions that, when executed by at least one processor, cause the at least one processor to: receive imaging data of a surgical site, wherein a robot arm is configured to position an end effector in the surgical site; generate a presentation to be presented on a user interface, wherein the presentation comprises: imaging data of the surgical site; and a map of an anatomy of the surgical site surrounding the imaging data; receive a user input on the user interface; and actuate the robot arm to move the end effector with a speed based on the user input.

Clause 68. A system, comprising: a surgical robot, comprising a robot arm configured to hold an end effector; and one or more processing devices configured to: receive an estimate of a position or a location of the end effector in a surgical site; receive an estimate of a depth of tissue in front of the end effector; generate a map of the surgical site, wherein the map extends beyond a field of view of real-time imaging data of the surgical site generated at a current position or location of the end effector; and present the map of the surgical site and the real-time imaging data on a display.

Clause 69. The system of clause 68, wherein the map of the surgical site shows a contour of a tissue of the surgical site.

Clause 70. The system of clause 68 or clause 69, wherein the real-time imaging data is positioned on the map at the current position or location of the end effector.

Clause 71. The system of any one of clauses 68-70, wherein the real-time imaging data is displayed in a border that indicates a direction and a magnitude of a force acting on the end effector.

Clause 72. The system of any one of clauses 68-71, wherein the one or more processing devices are further configured to: receive input on the display for moving the end effector; determine if the input is received in the map of the surgical site or in an end effector marker shown on the display; move the end effector at a first speed if the input is received in the map of the surgical site; and move the end effector at a second speed if the input is received in the end effector marker.

Clause 73. The system of any one of clauses 68-72, wherein the end effector marker is a fixed distance from the real-time imaging data on the display.

Clause 74. A system, comprising: a surgical robot, comprising a robot arm configured to hold an end effector; and one or more processing devices configured to; receive an estimate of a depth of tissue in front of the end effector; receive input to move the end effector in first and second directions; generate an instruction to move the end effector in first and second directions in accordance with the received input; and automatically generate an instruction to move the end effector in a third direction.

Clause 75. The system of clause 74, wherein: the first and second directions are parallel to a plane of the tissue in front of the end effector; and the third direction is perpendicular to the plane of the tissue in front of the end effector.

Clause 76. The system of clause 74 or clause 75, wherein the instruction to move the end effector in the third direction is to maintain a set distance between the end effector and the tissue in the third direction.

Based on the foregoing, it should now be understood that embodiments shown and described herein relate to processes and systems for an image-guide surgical robotic platform.

As utilized herein, the terms “comprise” and “comprising” are intended to be construed as being inclusive, not exclusive. As utilized herein, the terms “exemplary”, “example”, and “illustrative”, are intended to mean “serving as an example, instance, or illustration” and should not be construed as indicating, or not indicating, a preferred or advantageous configuration relative to other configurations. As utilized herein, the terms “about”, “generally”, and “approximately” are intended to cover variations that may existing in the upper and lower limits of the ranges of subjective or objective values, such as variations in properties, parameters, sizes, and dimensions. In one non-limiting example, the terms “about”, “generally”, and “approximately” mean at, or plus 10 percent or less, or minus 10 percent or less. In one nonlimiting example, the terms “about”, “generally”, and “approximately” mean sufficiently close to be deemed by one of skill in the art in the relevant field to be included. As utilized herein, the term “substantially” refers to the complete or nearly complete extend or degree of an action, characteristic, property, state, structure, item, or result, as would be appreciated by one of skill in the art. For example, an object that is “substantially.” circular would mean that the object is either completely a circle to mathematically determinable limits, or nearly a circle as would be recognized or understood by one of skill in the art. The exact allowable degree of deviation from absolute completeness may in some instances depend on the specific context. However, in general, the nearness of completion will be so as to have the same overall result as if absolute and total completion were achieved or obtained. The use of “substantially” is equally applicable when utilized in a negative connotation to refer to the complete or near complete lack of an action, characteristic, property, state, structure, item, or result, as would be appreciated by one of skill in the art. The use of the terminology X “or” Y herein should be interpreted as meaning either “X” or “Y” individually, or both “X and Y” together.

All patents and publications mentioned in this specification are herein incorporated by reference to the same extent as if each independent patent and publication was specifically and individually indicated to be incorporated by reference.

Numerous modifications and alternative embodiments of the present disclosure will be apparent to those skilled in the art in view of the foregoing description. Accordingly, this description is to be construed as illustrative only and is for the purpose of teaching those skilled in the art the best mode for carrying out the present disclosure. Details of the structure may vary substantially without departing from the spirit of the present disclosure, and exclusive use of all modifications that come within the scope of any appended claims is reserved. Within this specification, embodiments have been described in a way which enables a clear and concise specification to be written, but it is intended and will be appreciated that embodiments may be variously combined or separated without parting from the scope of the present disclosure. It is intended that the present disclosure be limited only to the extent required by any appended claims and the applicable rules of law.

1. A system, comprising:
a surgical robot, comprising a robot arm configured to hold an end effector; and
at least one circuit configured to:
generate a presentation to be presented on a user interface, wherein the presentation comprises:
imaging data of a surgical site;
a map of an anatomy of the surgical site surrounding the imaging data; and
a first designated touch area;
receive a user input on the user interface; and
actuate the robot arm to move the end effector in one of a depth direction or a planar direction based on the user input.

2. The system of claim 1, wherein the surgical site comprises a prostate.

3. The system of claim 1, wherein the at least one circuit is configured to actuate the robot arm to move the end effector in one of a depth direction or a planar direction based on a location of the received user input on the user interface.

4. The system of claim 1, wherein, when the received user input is in the first designated touch area, the at least one circuit is configured to actuate the robot arm to move the end effector in the depth direction based on the user input received in the first designated touch area.

5. The system of claim 4, wherein the at least one circuit is configured to actuate the robot arm to move the end effector in the depth direction with a speed based on a speed of the user input received in the first designated touch area.

6. The system of claim 4, wherein:
the received user input comprises one or more contact points with the user interface; and
the at least one circuit is configured to actuate the robot arm to move the end effector in the depth direction with a speed based on the one or more contact points.

7. The system of claim 6, wherein the at least one circuit is configured to:
actuate the robot arm to move the end effector in the depth direction with a first speed when the user input comprises one contact point; and
actuate the robot arm to move the end effector in the depth direction with a second speed when the user input comprises a plurality of contact points.

8. The system of claim 4, wherein, when the received user input is outside the first designated touch area, the at least one circuit is configured to actuate the robot arm to move the end effector in the planar direction based on the user input received outside the first designated touch area.

9. The system of claim 8, wherein the at least one circuit is configured to actuate the robot arm to move the end effector in the planar direction with a speed based on a speed of the user input received outside the first designated touch area.

10. The system of claim 8, wherein:
the received user input comprises one or more contact points with the user interface; and
the at least one circuit is configured to actuate the robot arm to move the end effector in the planar direction with a speed based on the one or more contact points.

11. The system of claim 10, wherein the at least one circuit is configured to:
actuate the robot arm to move the end effector in the planar direction with a first speed when the user input comprises one contact point; and
actuate the robot arm to move the end effector in the planar direction with a second speed when the user input comprises a plurality of contact points.

12. The system of claim 8, wherein the received user input is in a second designated touch area, wherein the second designated touch area comprises at least a portion of the imaging data and at least a portion of the map.

13. The system of claim 1, wherein, when the received user input is in at least one of the imaging data or the map, the at least one circuit is configured to actuate the robot arm to move the end effector in the planar direction based on the user input received in the at least one of the imaging data or the map.

14. The system of claim 13, wherein the at least one circuit is configured to actuate the robot arm to move the end effector in the planar direction with a speed based on a speed of the user input received in at least one of the imaging data or the map.

15. The system of claim 13, wherein:
the received user input comprises one or more contact points with the user interface; and
the at least one circuit is configured to actuate the robot arm to move the end effector in the planar direction with a speed based on the one or more contact points.

16. The system of claim 15, wherein the at least one circuit is configured to:
actuate the robot arm to move the end effector in the planar direction with a first speed when the user input comprises one contact point; and
actuate the robot arm to move the end effector in the planar direction with a second speed when the user input comprises a plurality of contact points.

17. The system of claim 13, wherein the at least one circuit is configured to:
actuate the robot arm to move the end effector in the planar direction with a first speed when the user input is received in the map; and
actuate the robot arm to move the end effector in the planar direction with a second speed when the user input is received in the imaging data.

18. The system of any one of claims 1, wherein the planar direction is defined in a plane, and the depth direction is perpendicular to the plane.

19. The system of any one of claims 1, wherein the user input is a swiping gesture on the user interface.

20. The system of claim 1, wherein the at least one circuit is configured to actuate the robot arm to move the end effector in one of a depth direction or a planar direction based on a location of the user input received on the user interface and a gesture of the user input received on the user interface.

21. The system of claim 1, wherein the at least one circuit is configured to:
actuate the robot arm to move the end effector in the depth direction when the received user input is a first gesture received in the first designated touch area;
actuate the robot arm to move the end effector in the planar direction when the received user input is the first gesture received outside the first designated touch area; and
actuate the robot arm to move the end effector in the depth direction when the received user input is a second gesture received outside the first designated touch area.

22. The system of claim 21, wherein the at least one circuit is configured to actuate the robot arm to move the end effector in the depth direction at a speed for fine movement when the received user input is a second gesture received outside the first designated touch area.

23. The system of claim 21, wherein the planar direction is defined in a plane, and the depth direction is perpendicular to the plane.

24. The system of claim 21, wherein the first gesture is a swiping gesture on the user interface, and wherein the second gesture is a pinching gesture on the user interface.

25. A system, comprising:
a surgical robot, comprising a robot arm configured to hold an end effector; and
at least one circuit configured to:
generate a presentation to be presented on a user interface, wherein the presentation comprises:
imaging data of a surgical site; and
a map of an anatomy of the surgical site surrounding the imaging data;
receive a user input on the user interface; and
actuate the robot arm to move the end effector with a speed based on the user input.

26-33. (canceled)

34. A method, comprising:
receiving imaging data of a surgical site, wherein a robot arm is configured to position an end effector in the surgical site;
generating a presentation to be presented on a user interface, wherein the presentation comprises:
imaging data of the surgical site;
a map of an anatomy of the surgical site surrounding the imaging data; and
a first designated touch area;
receiving a user input on a user interface; and
actuating the robot arm to move the end effector in one of a depth direction or a planar direction based on the user input.

35-56. (canceled)

57. A method, comprising:
receiving imaging data of a surgical site, wherein a robot arm is configured to position an end effector in the surgical site;
generating a presentation to be presented on a user interface, wherein the presentation comprises:
imaging data of the surgical site; and
a map of an anatomy of the surgical site surrounding the imaging data;
receiving a user input on the user interface; and
actuating the robot arm to move the end effector with a speed based on the user input.

58-65. (canceled)

66. At least one non-transitory computer-readable storage medium having encoded thereon executable instructions that, when executed by at least one processor, cause the at least one processor to:
receive imaging data of a surgical site, wherein a robot arm is configured to position an end effector in the surgical site;
generate a presentation to be presented on a user interface, wherein the presentation comprises:
imaging data of the surgical site;
a map of an anatomy of the surgical site surrounding the imaging data; and
a first designated touch area;
receive a user input on a user interface; and
actuate the robot arm to move the end effector in one of a depth direction or a planar direction based on the user input.

67. At least one non-transitory computer-readable storage medium having encoded thereon executable instructions that, when executed by at least one processor, cause the at least one processor to:
receive imaging data of a surgical site, wherein a robot arm is configured to position an end effector in the surgical site;
generate a presentation to be presented on a user interface, wherein the presentation comprises:
imaging data of the surgical site; and
a map of an anatomy of the surgical site surrounding the imaging data;
receive a user input on the user interface; and
actuate the robot arm to move the end effector with a speed based on the user input.

68-76. (canceled)
